(19) United States
(12) Patent Application Publication
(10) Pub. No.: US 2010/0061995 A1
(43) Pub. Date: Mar. 11, 2010

(54) Immunotherapy To Treat Or Prevent Viral Infection

(76) Inventors: Damian Michael Carragher, Oldham (GB); Denise Ann Kaminski, Rochester, NY (US); Frances E. Lund, Rochester, NY (US); Troy Douglas Randall, Rochester, NY (US)

Correspondence Address: MEDLEN & CARROLL, LLP, Suite 350, 101 Howard Street, San Francisco, CA, 94105, US

(21) Appl. No.: 12/206,157

(22) Filed: Sep. 8, 2008

(51) Int. Cl. A61K 39/395; A61P 31/16; A61K 31/7088

(52) U.S. Cl. 424/147.1; 424/159.1; 514/44 R

(57) ABSTRACT

The prevention and treatment of influenza is described, by using passive immunization with antibodies (e.g. polyclonal, monoclonal, etc.) to one or more conserved influenza proteins. Both antibodies and fragments thereof are contemplated, raised against conserved proteins such as nucleoproteins.

### FIELD OF THE INVENTION

The field of the invention relates to the prevention and treatment of disease, and more particularly, the prevention and treatment of viral infection (e.g. influenza), by using passive immunization with antibodies to one or more conserved viral proteins, including but not limited to conserved influenza viral proteins.

### BACKGROUND OF THE INVENTION

The 1918 pandemic is estimated to have killed at least twenty million people worldwide. It was caused by a particular influenza viral strain and was characterized by both rapid transmission and severe symptoms. Today there is a concern among scientists about a similar pandemic, in this case from an avian influenza virus. The Senior United Nations System Coordinator for Avian and Human Influenza has warned that an outbreak of avian influenza could kill anywhere between 5 million and 150 million people.

Even without a pandemic, influenza infection presents both a health risk and health cost. On average, 5% to 20% of the U.S. population gets influenza (commonly called “the flu”) each year. More than 100,000 people are hospitalized from flu complications, and approximately 36,000 people die. Some people, such as older people, young children, and people with certain health conditions (e.g. immunocompromised people), are at high risk for serious flu complications.

Influenza A and B viruses are responsible for seasonal flu epidemics each year. Over the course of a flu season, different types (A & B) and subtypes of influenza A viruses can circulate through the population and cause illness. A particular problem for treatment strategies is the fact that influenza viruses are constantly changing through a process called “antigenic drift.” Thus, a vaccine that might have been useful last year may be less effective or ineffective this year.

What is needed is a treatment strategy that avoids the problem of antigenic drift.

### SUMMARY OF THE INVENTION

The present invention contemplates generating antibody to conserved viral proteins such as nucleoproteins and RNA-binding proteins from viruses [e.g. HIV, metapneumovirus, coronavirus (which causes SARS), etc.] dangerous to man and animals (i.e. pathogenic viruses), so that the antibody can be used for passive immunization. In one embodiment, the present invention contemplates administering (prior to or after infection) antibodies (or portions or fragments thereof) raised against a conserved protein (or portion thereof) of an Influenza virus (including avian and mammalian isolates). In one embodiment, antibodies are generated to conserved internal proteins or internal protein domains of influenza. In one embodiment, the conserved protein (or portion thereof) is other than hemagglutinin (HA) and neuraminidase (NA), both of which are known to vary due to antigenic drift. In one embodiment, the conserved Influenza viral protein is selected from the group consisting of: PB1, PB2, PA, NS1, NS2, M1, M2 and PB1-F2. In a particularly preferred embodiment, the antibodies are generated against recombinant nucleoprotein (NP) from influenza virus preferably, purified soluble recombinant NP). In one embodiment, the antibodies are polyclonal. In another embodiment, the antibodies are monoclonal. In yet another embodiment, the antibodies are human or humanized.

While influenza is emphasized herein, it is not intended that the present invention be limited to the particular virus. Thus, in one embodiment, the present invention contemplates a method for treating a viral infection (e.g. HIV, metapneumovirus, coronavirus etc.) comprising: a) providing: i) a subject exhibiting symptoms of (or at risk for) a viral infection, ii) a composition comprising antibody or fragment thereof reactive with a conserved viral protein (e.g. a viral nucleoprotein); b) administering said composition to said subject under conditions such that said symptoms are reduced.

In one embodiment, the present invention contemplates a method for treating a viral infection comprising: a) providing: i) a mammal exhibiting symptoms of a viral infection, ii) a composition comprising antibody reactive with influenza viral nucleoprotein; b) administering said composition to said mammal under conditions such that said symptoms are reduced. It is not intended that the present invention be limited by the nature of the virus. In one embodiment, said viral infection is infection by Influenza A (including strains of avian, swine, equine and human origin). While treatment of non-humans is also contemplated, the preferred subject is a human, including but not limited to a child (10 years in age or less, more typically 7 years in age or less, and commonly 5 years in age or less), the elderly (60 years of age or greater, more typically 65 years of age or greater, most commonly 70 years to 100 years of age), and the immunocompromised (e.g. by age or disease such as AIDS, or because of steroids or other anti-inflammatory drugs taken to treat a condition, such as an autoimmune conditions like Crohn's disease, or because of anti-proliferative drugs taken to treat a condition, such as cancer). It is not intended that the present invention be limited by the nature of the antibody (e.g. polyclonal, monoclonal, human, or humanized) or that only intact antibody be used (since antibody fragments are also functional) or that only unpurified antibody be used (since antibody can be readily purified by a number of means described below and used for passive immunization in the same manner). Where the antibody is polyclonal, it can be raised a number of ways, including raised in a non-human mammal immunized with influenza viral nucleoprotein, and raised in a non-mammal (e.g. a bird, as described below) immunized with influenza viral nucleoprotein.

In one embodiment, the present invention contemplates a method for treating a high risk viral infection comprising: a) providing: i) a mammal exhibiting symptoms of a viral infection, said mammal at higher risk for developing complications than the normal population, ii) a composition comprising antibody reactive with influenza viral nucleoprotein; b) administering said composition to said mammal under conditions such that said symptoms are reduced. Those at high risk for complications include people 65 years or older, people with chronic medical conditions (such as asthma, diabetes, or heart disease), pregnant women, and young children. Complications include bacterial pneumonia, dehydration, and worsening of chronic medical conditions, such as congestive heart failure, asthma or diabetes, and death (e.g. due to dehydration or worsening of the chronic medical condition, or simply due to the weakening effect of infection). Children and adults may develop sinus problems and ear infections. Again, it is not intended that the present invention be limited by the nature of the virus. In one embodiment, said viral infection is infection by Influenza A (including strains of avian, swine, equine and human origin).

In one embodiment, the present invention contemplates a method for treating a viral infection demonstrating drug resistance comprising: a) providing: i) a mammal exhibiting symptoms of a viral infection, said viral infection showing drug resistance (e.g. in vitro or in vivo), ii) a composition comprising antibody reactive with influenza viral nucleoprotein; b) administering said composition to said mammal under conditions such that said symptoms are reduced. It is not intended that the present invention be limited by the type of drug resistance. In one embodiment, the virus is resistant to standard treatment with oseltamivir. It is not intended that the present invention limited by some time period during which treatment with standard anti-viral drugs fails. In one embodiment, passive immunization with the herein described antibodies to NP is performed after just 6-12 hours of standard drug therapy (but more typically 12-24 hours, and even 24-72 hours). In one embodiment, the virus is isolated from the infected subject and tested for drug resistance in vitro (in such a case the subject may or may not have been treated in vivo with a drug). Again, it is not intended that the present invention be limited by the nature of the virus. In one embodiment, said viral infection is infection by Influenza A (including strains of avian, swine, equine and human origin).

In one embodiment, the present invention contemplates a method for treating a viral infection suspected of being caused by a drug resistant strain comprising: a) providing: i) a mammal exhibiting symptoms of a viral infection, said viral infection suspected to be drug resistant (e.g. suspected because the progression of disease in the subject who has been treated with drugs, or from failed drug treatment in other people from whom it is believed the virus spread to the subject, from molecular analysis of the isolate from the subject or from other people, or from the extent of the spread of infection in a hospital or other situation or region, etc.), ii) a composition comprising antibody reactive with influenza viral nucleoprotein; b) administering said composition to said mammal under conditions such that said symptoms are reduced. Again, it is not intended that the present invention be limited by the nature of the virus. In one embodiment, said viral infection is infection by Influenza A (including strains of avian, swine, equine and human origin).

In one embodiment, the present invention contemplates a method for treating a viral infection comprising: a) providing: i) a mammal exhibiting symptoms of a viral infection, ii) a first composition comprising antibody (or fragment thereof) reactive with influenza viral nucleoprotein; and iii) a second composition comprising an anti-viral drug; b) administering said first and second compositions to said mammal under conditions such that said symptoms are reduced. It is not intended that the present invention be limited by the precise approach to administering in step b). For example, the first and second compositions can be administered in a mixture together and thus simultaneously. On the other hand, they can be administered in series (in any order) and there may or may not be a time period (e.g. minutes, hours, days) between each administration. The present invention contemplates, in one embodiment, the mixture as a composition of matter. It is not intended that the present invention be limited to the particular drug. In one embodiment, the drug is Oseltamivir. In another embodiment, the drug is Zanamivir. In yet another embodiment, the second composition comprises two anti-viral drugs.

In one embodiment, the present invention contemplates a method for treating a viral infection comprising: a) providing: i) a mammal exhibiting symptoms of a viral infection, wherein said mammal is not suited for treatment with an anti-viral drug; ii) a composition comprising antibody reactive with influenza viral nucleoprotein; b) administering said first and second compositions to said mammal under conditions such that said symptoms are reduced. It is not intended that the present invention be limited to the nature of the mammal or the condition that makes the mammal not suitable for treatment with anti-viral drugs. In one embodiment, the mammal is pregnant. In one embodiment, the mammal is a child or is elderly. In one embodiment, the mammal is a higher risk (than the general population) for side effects. In one embodiment, the subject has underlying lung disease such as asthma and chronic obstructive pulmonary disease.

The present invention also contemplates, in one embodiment, a method for treating an Influenza A viral infection comprising: a) providing: i) a human exhibiting symptoms of an Influenza A viral infection, ii) a composition comprising antibody reactive with Influenza A viral nucleoprotein; b) administering said composition to said human under conditions such that said symptoms are reduced. In one embodiment, said Influenza A viral infection is from an avian, swine, equine or human strain. In one embodiment, the human is a child, an elderly human, or an immunocompromised human.

The present invention, in one embodiment, contemplates a method for protecting against an Influenza A viral infection comprising: a) providing: i) a human at risk for an Influenza A viral infection (e.g. because of infection in others around the human, because the human works in a hospital, nursing home or other health related institution, because of an outbreak in city, state or country, because of travel to a region known to have a high rate of infection, etc.), ii) a composition comprising antibody reactive with Influenza A viral nucleoprotein; b) administering said composition to said human prior to any symptoms of infection. In one embodiment, said Influenza A viral infection is from an avian, swine, equine or human strain. In one embodiment, the human is a child, an elderly human, or an immunocompromised human.

In one embodiment, the present invention contemplates a method for treating a first human exposed to a second human, said second human having a viral infection comprising: a) providing: i) a first human, exposed to a second human (e.g. where the second human is a patient and the first human is a nurse, doctor, or other health worker; where first and second humans otherwise came into contact, e.g. because they are family members or traveled together, or interact during child care or elder care, or where the first human is in the military, e.g. because of exposure to the second human during combat, troop housing, troop movements and the like, or where the second human is in the military and has been subjected to a “weaponized” flu) ii) a second human exhibiting symptoms of a viral infection, iii) a composition comprising antibody reactive with influenza viral nucleoprotein; b) administering said composition to said first human. In one embodiment, said first human is not infected. In another embodiment, said first human is infected but shows no symptoms. In another embodiment, said first human is infected and is beginning to show symptoms. In one embodiment, said viral infection is an Influenza A viral infection from an avian, swine, equine or human strain. In one embodiment, said first human is a child, an elderly human, or an immunocompromised human.

Because NP is highly conserved among influenza A viruses, the use of this protein to generate antibodies for passive immunization (discussed in more detail below) will enhance protection against many different types of influenza virus. It is contemplated in one embodiment, that this type of treatment may help to protect the public from seasonal as well as avian/pandemic influenza. In a particularly preferred embodiment, it is contemplated that such antibodies (or fragments thereof) will help to reduce the influenza-related morbidity and mortality the elderly, who are particularly susceptible to this disease.

Without intending to limit the invention in any manner to a mechanism, it is believed that: 1) antibody production in the NP-vaccinated animal is essential for protection, and 2) immune serum (comprising antibody) from NP-vaccinated mice can passively transfer protection to naive recipients in an antibody-dependent manner.

However, antibody production (for passive immunization) is also contemplated for other conserved proteins in the above-specified embodiments. For example, antibody production to NS1 protein of a variety of influenza viruses, included avian flu. The NS1 protein of the highly pathogenic avian H5N1 viruses circulating in poultry and waterfowl in Southeast Asia is currently believed to be responsible for the enhanced virulence of the strain. H5N1 NS1 is characterized by a single amino acid change at position 92. By changing the amino acid from glutamic acid to aspartic acid, researchers were able to annul the effect of the H5N1 NS1. This single amino acid change in the NS1 gene greatly increased the pathogenicity of the H5N1 influenza virus. By raising antibody to NS1 protein (such as the NS1 protein of avian H5N1 virus), the present invention contemplates protective antibody that can be used in passive immunization therapy for man and animals (including birds).

In any of the above-mentioned embodiments, the present invention contemplates administering vector(s) instead of antibody, e.g. genetic passive immunotherapy by administration of vectors (e.g. Ad, Ad-like and AAV vectors) encoding a specific monoclonal antibody to a conserved viral protein, e.g. NP. Thus, in one embodiment, the present invention contemplates a method for treating a viral infection comprising: a) providing: i) a subject exhibiting symptoms of a viral infection, ii) a composition comprising a vector encoding antibody or an antibody fragment reactive with a conserved viral protein (e.g. a viral nucleoprotein); b) administering said composition to said subject under conditions such that antibody is produced in said subject and said symptoms are reduced. It is not intended that the present invention be limited by the nature of the vector or the particular placement of elements. Nonetheless, in one embodiment, said vector comprises antibody light-chain and heavy-chain nucleic acid sequences linked with an internal ribosome entry site and constructed into an adenoviral vector under the control of a promoter.

In a preferred embodiment, the present invention contemplates antibodies reactive with a conserved internal viral protein or internal domain of a viral protein. Thus, in one embodiment, the present invention contemplates a method for treating a viral infection comprising: a) providing: i) a subject exhibiting symptoms of a viral infection, ii) a composition comprising a vector encoding antibody or an antibody fragment reactive with a conserved internal viral protein (e.g. an internal domain of M2); b) administering said composition to said subject under conditions such that antibody is produced in said subject and said symptoms are reduced. It is not intended that the present invention be limited by the nature of the vector or the particular placement of elements. Nonetheless, in one embodiment, said vector comprises antibody light-chain and heavy-chain nucleic acid sequences linked with an internal ribosome entry site and constructed into an adenoviral vector under the control of a promoter.

### Definitions

All viruses with negative-sense RNA genomes encode a single-strand RNA-binding “nucleoprotein” (NP). The primary function of NP is to encapsidate the virus genome for the purposes of RNA transcription, replication and packaging. Influenza virus NP is a well-studied example. The present invention contemplates NP from a variety of sources, including from avian and mammalian viral isolates.

As noted above, the present invention contemplates generating antibodies against conserved proteins of viruses. In one embodiment, the present invention contemplates generating antibodies against conserved “internal” proteins or “internal” protein domains of viruses (such as influenza). The term “internal” is used herein to distinguish from the surface exposed proteins or protein domains of viruses. For example, in one embodiment, the present invention contemplates generating antibody to an “internal” domain of M2 (as distinguished from the surface exposed domain of M2).

“PB1-F2” is produced by infected cells (although not incorporated into the virion) and is translated by an alternate reading frame from the PB1 segment.

Infected subjects develop “symptoms” of infection. Influenza usually starts suddenly and may include the following symptoms: fever (usually high), headache, tiredness (can be extreme), cough, sore throat, runny or stuffy nose, body aches, diarrhea and vomiting (more common among children than adults). The flu can cause mild to severe illness and at times can lead to death. More severe symptoms include dehydration and body weight loss. It is not intended that passive immunization with the antibodies herein described completely eliminate all symptoms. It is sufficient that one or more symptoms (e.g. body weight loss) are reduced in intensity or that the overall duration of disease symptoms be reduced in time. It is also sufficient if the treated subject presents with fewer symptoms (e.g. no dehydration).

Those at “high risk for complications” include people 60-65 years or older, people with chronic medical conditions (such as asthma, diabetes, or heart disease), pregnant women, and young children. Complications include bacterial pneumonia, dehydration, and worsening of chronic medical conditions, such as congestive heart failure, asthma or diabetes, and death. Children and adults may develop sinus problems and ear infections.

The flu usually spreads from person to person in respiratory droplets when people who are infected cough or sneeze. People occasionally may become infected by touching something with influenza virus on it and then touching their mouth, nose or eyes. Healthy adults may be able to infect others 1 day before getting symptoms and up to 5 days after getting sick. Therefore, it is possible to give someone the flu before you know you are sick as well as while you are sick. Therefore, it is possible to treat someone with antibodies (passive immunization) before they know they are sick as well as while they are sick. Thus, in one embodiment, the present invention contemplates passive immunization of members of a population (e.g. workers in a hospital, people within a city where there is an outbreak, members of the military) in order to reduce the spread of infection (or reduce the rate that the infection spreads). In one embodiment, subjects are given antibodies to NP (passive immunization) prophylactically to prevent infection, reduce the incidence of infection, or at least reduce morbidity and mortality upon infection.

As used herein “immunoglobulin” refers to any of a group of large glycoproteins that are secreted by plasma cells and that function as antibodies in the immune response by binding with specific antigens. There are five classes of immunoglobulins: IgA, IgD, IgE, IgG, and IgM.

The term “antibody,” as used herein, is intended in one embodiment to refer to immunoglobulin molecules comprised of four polypeptide chains, two heavy (H) chains and two light (L) chains (lambda or kappa) inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each variable region (VH or VL) contains 3 CDRs, designated CDR1, CDR2 and CDR3. Each variable region also contains 4 framework sub-regions, designated FR1, FR2, FR3 and FR4.

As used herein, the term “antibody fragments” refers to a portion of an intact antibody. Examples of antibody fragments include, but are not limited to, linear antibodies, single-chain antibody molecules, Fv, Fab and F(ab′)₂ fragments, and multispecific antibodies formed from antibody fragments. The antibody fragments preferably retain at least part of the heavy and/or light chain variable region.

As used herein, the terms “complementarity determining region” and “CDR” refer to the regions that are primarily responsible for antigen-binding. There are three CDRs in a light chain variable region (CDRL1, CDRL2, and CDRL3), and three CDRs in a heavy chain variable region (CDRH1, CDRH2, and CDRH3). The particular designation in the art for the exact location of the CDRs varies depending on what definition is employed. Preferably, the IMGT designations are used, which uses the following designations for both light and heavy chains: residues 27-38 (CDR1), residues 56-65 (CDR2), and residues 105-116 (CDR3); see Lefrance, M P, The Immunologist, 7:132-136, 1999, herein incorporated by reference. The residues that make up the six CDRs have also been characterized by Kabat and Chothia as follows: residues 24-34 (CDRL1), 50-56 (CDRL2) and 89-97 (CDRL3) in the light chain variable region and 31-35 (CDRH1), 50-65 (CDRH2) and 95-102 (CDRH3) in the heavy chain variable region; Kabat et al., (1991) Sequences of Proteins of Immunological Interest, 5th Ed. Public Health Service, National Institutes of Health, Bethesda, Md., herein incorporated by reference; and residues 26-32 (CDRL1), 50-52 (CDRL2) and 91-96 (CDRL3) in the light chain variable region and 26-32 (CDRH1), 53-55 (CDRH2) and 96-101 (CDRH3) in the heavy chain variable region; Chothia and Lesk (1987) J. Mol. Biol. 196: 901-917, herein incorporated by reference. Unless otherwise specified, the terms “complementarity determining region” and “CDR” as used herein, include the residues that encompass IMGT, Kabat and Chothia definitions. Also, unless specified, as used herein, the numbering of CDR residues is according to IMGT.

As used herein, the term “framework” refers to the residues of the variable region other than the CDR residues as defined herein. There are four separate framework sub-regions that make up the framework: FR1, FR2, FR3, and FR4. In order to indicate if the framework sub-region is in the light or heavy chain variable region, an “L” or “H” may be added to the sub-region abbreviation (e.g., “FRL1” indicates framework sub-region 1 of the light chain variable region). Unless specified, the numbering of framework residues is according to IMGT.

As used herein, “humanized” forms of non-human (e.g., murine) antibodies are antibodies that contain minimal sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are generally made to further refine antibody performance. In general, the humanized antibody will comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody may also comprise at least a portion of an immunoglobulin constant region (Fc), typically that of a human immunoglobulin. Examples of methods used to generate humanized antibodies are described in U.S. Pat. No. 5,225,539 to Winter et al. (herein incorporated by reference).

Importantly, early methods for humanizing antibodies often resulted in antibodies with lower affinity than the non-human antibody starting material. More recent approaches to humanizing antibodies address this problem by making changes to the CDRs. See U.S. Patent Application Publication No. 20040162413, hereby incorporated by reference. In some embodiments, the present invention provides an optimized heteromeric variable region (e.g. that may or may not be part of a full antibody or other molecule) having equal or higher antigen binding affinity than a donor heteromeric variable region, wherein the donor heteromeric variable region comprises three light chain donor CDRs, and wherein the optimized heteromeric variable region comprises: a) a light chain altered variable region comprising; i) four unvaried human germline light chain framework regions, and ii) three light chain altered variable region CDRs, wherein at least one of the three light chain altered variable region CDRs is a light chain donor CDR variant, and wherein the light chain donor CDR variant comprises a different amino acid at only one, two, three or four positions compared to one of the three light chain donor CDRs (e.g. the at least one light chain donor CDR variant is identical to one of the light chain donor CDRs except for one, two, three or four amino acid differences).

As used herein, the terms “subject” and “patient” refer to any animal, such as a bird, or such as a mammal like a dog, cat, livestock, and preferably a human.

### DESCRIPTION OF THE FIGURES

FIG. 1 shows an alignment of the amino acid sequences (SEQ ID NOS: 1-49) of various sources of influenza nucleoprotein.

FIG. 2 shows a designed nucleic acid sequence (SEQ ID NO:50) encoding the NP (with some amino acid changes) of strain PR834.

FIG. 3 shows a coding sequence (SEQ ID NO:52) for NP from a Taiwan strain of avian Influenza A virus.

FIG. 4 shows a coding sequence (SEQ ID NO:54) for NP from an Indonesian strain of avian Influenza A virus.

FIG. 5 shows a coding sequence (SEQ ID NO:56) for NP from a Hong Kong strain of avian Influenza A virus.

FIG. 6 is a bar graph showing anti-NP antibody titers by ELISA.

FIG. 7 shows lung viral titers after control (LPS) and test (NP/LPS) immunizations, demonstrating protection against viral challenge after active immunization.

FIG. 8 shows lung viral titers in recipient animals after passive immunization with serum from donor animals receiving control (LPS) and test (NP/LPS) immunizations, demonstrating protection against viral challenge after passive immunization with donor antibody.

FIG. 9 shows a coding sequence (SEQ ID NO:57) for NP from human metapneumovirus.

FIG. 10 shows a coding sequence (SEQ ID NO:58) for NP from human coronavirus.

FIG. 11 shows the amino acid sequence (SEQ ID NO:59) for an HIV gene product which is cleaved into several products that include RNA-binding proteins. In one embodiment, the present invention contemplates such RNA-binding proteins as immunogens for raising antibody useful in passive immunization. For example, the nucleocapsid has the amino acid sequence mqrgnfrnqr kivkcfncgk eghtarncra prkkgcwkcg keghqmkdct erqan (SEQ ID NO:60) and the matrix protein sequence is: mgarasvlsg geldrwekir lrpggkkkyk lkhivwasre lerfavnpgl letsegcrqi lgqlqpslqt gseelrslyn tvatlycvhq rieikdtkea ldkieeeqnk skkkaqqaaa dtghsnqvsq ny (SEQ ID NO:61).

FIG. 12 shows a coding sequence (SEQ ID NO:62) for matrix protein 1 (M1) and M2 of an Influenza A virus (A/Puerto Rico/8/1934(H1N1)).

FIG. 13 shows the amino acid sequence (SEQ ID NO:63) for NS1 of an Influenza A virus (A/Puerto Rico/8/1934(H1N1)).

FIG. 14 shows the amino acid sequence (SEQ ID NO:64) for NS2 of an Influenza A virus (A/Puerto Rico/8/1934(H1N1)).

FIG. 15 shows the amino acid sequence (SEQ ID NO:65) for PA of an Influenza A virus (A/Puerto Rico/8/1934(H1N1)).

FIG. 16 shows the amino acid sequence (SEQ ID NO:66) for PB1 of an Influenza A virus (A/Puerto Rico/8/1934(H1N1)).

FIG. 17 shows the amino acid sequence (SEQ ID NO:67) for PB2 of an Influenza A virus (A/Puerto Rico/8/1934(H1N1)).

FIG. 18 shows the amino acid sequence (SEQ ID NO:68) for M2 of an Influenza A virus (A/Puerto Rico/8/1934(H1N1)).

### DESCRIPTION OF THE INVENTION

In one embodiment, the present invention contemplates active immunization with a conserved viral protein, including but not limited to a conserved influenza protein such as NP. In another embodiment, the present invention contemplates passive immunization with antibody specific to a conserved influenza protein such as NP. NP is offered here simply as an example of the larger set of conserved viral proteins discussed above.

A. Active Immunization

Immunization with HA and/or NA generates a strong immune response. However, these proteins vary considerably among strains (indeed, strains are identified based on this variability). Moreover, as discussed above, they exhibit antigenic drift.

Vaccines based on these proteins offer the opportunity for protection in high risk groups such as the elderly. However, the availability of such vaccines to the whole population is problematic. First, there is the question of the cost to immunize an entire population where a relatively small percentage of that population will be exposed (absent the emergence of a pandemic strain). Second, there is the problem of effectiveness since these antigens are constantly changing.

Both natural infection with influenza virus and vaccination with recombinant NP elicit NP-specific antibodies. Sukeno et al. Tohoku J. Exp. Med. 128:241-249. Rangel-Moreno et al. J. Immunol. 180:454-463. However, anti-NP antibodies have been considered to be ineffective because they have been reported not to neutralize virus, and because passive transfer of such antibodies does not protect naive immunodeficient scid recipient mice. Gerhard et al. Immunol. Rev. 159:95-103.

In one embodiment, so-called DNA vaccines are employed whereby a recipient receives an expression vector expressing NP protein under the control of a promoter.

B. Passive Immunization

Because of the problems with active immunization, the present invention contemplates that the preferred treatment comprises passive immunization. Passive immunization, like active immunization, relies on antibodies binding to antigens. Typically, in the case of passive immunization, the antibody used to bind antigen is not made in the animal afflicted with the disease. In one embodiment of the present invention, an immune response is generated in a first animal (which can be a human or non-human). The serum (or purified antibody fraction thereof) of the first animal is then administered to the afflicted animal (typically, a human, but non-human treatment is also contemplated) to supply a source of specific and reactive antibody. Without limiting the present invention in any manner by the mechanism by which treatment is effective, it is believed that the administered antibody functions to some extent as though it were endogenous antibody, i.e. antibody raised by vaccination (by way of example).

In some situations, for example, where the subject is pregnant and vaccination raises risks, passive immunization may be the only appropriate treatment. Moreover, where the subject has been vaccinated, but nonetheless becomes infected and shows symptoms, passive immunization may be the only treatment immediately available that will reduce morbidity and the risk of mortality. Therefore, the present invention, in one embodiment, contemplates passive immunization with the herein described antibodies in infected people that have been previously vaccinated (e.g. with an ineffective or less than optimal vaccine).

This is particularly true given the increasing drug resistance of influenza viral strains (including viral strains thought to be potential sources of pandemics). For example, in 2005, there was a report of the isolation of an H5N1 virus (avian flu) from a Vietnamese girl that is resistant to the drug oseltamivir. See Nature 437, 1108 (20 Oct. 2005). Therefore, the present invention, in one embodiment, contemplates passive immunization with the herein described antibodies in people infected with drug resistant strains or strains believed to be drug resistant or strains believed to be potential sources of pandemics (e.g. avian flu). In addition, where there is an outbreak of a viral strain known to be drug resistant, the present invention, in one embodiment, contemplates passive immunization with the herein described antibodies in people not yet infected (prophylactic treatment by passive immunization).

Even where the viral strain is not drug resistant, some drugs are not appropriate for some individuals, e.g. pregnant and very young children. For example, Zanamivir is approved to treat flu in people 7 years and older and to prevent flu in people 5 years and older. Therefore, the present invention, in one embodiment, contemplates passive immunization with the herein described antibodies in people under 7 and under 5 years of age, for treatment and prevention, respectively.

Even where the viral strain is not drug resistant, some drugs exhibit side effects such that they should not be given to certain patient groups. For example, Zanamivir is generally not recommended for use in persons with underlying lung disease such as asthma and chronic obstructive pulmonary disease. Therefore, the present invention, in one embodiment, contemplates passive immunization with the herein described antibodies in people with asthma and pulmonary disease. As another example, oseltamivir has recently been associated with neuropsychiatric side effects (confusion with risks of self-injury, particularly in children). Therefore, the present invention, in one embodiment, contemplates passive immunization with the herein described antibodies in people at risk for or exhibiting such side effects.

1. Raising Antibody. In one embodiment, the first step in treatment by passive immunization involves raising an antibody with reactivity that is specific for the conserved influenza viral protein (or portion thereof). In one embodiment, the present invention contemplates administering polyclonal antibody specific for a conserved viral protein (e.g. NP) to humans, where the antibody has been raised in an animal.
i) Polyclonal Antibodies Raised In Animals

In one embodiment, the present invention contemplates raising polyclonal antibodies to NP in horses. Horses are sturdy and tolerant to the antibody-raising process. Most importantly, they yield large volumes of blood (as much as ten liters per bleeding for large animals).

There are some disadvantages, however, when using horses for antibody production. First, for large production of antibodies, horses more than 5 years old and usually less than 8 years old are required. Second, production should be under veterinary care and supervision. Third, tetanus is known to be a common disease among horses; animals must be immunized as soon as they are introduced to the farm. Fourth, large amounts of antigen are required for immunization in order to generate a satisfactory immune response in horses. Fifth, horse antibody binds and activates human and other mammalian complement pathways, leading (at the very least) to complement depletion and (at worst) to a more acute reaction by the host. Sixth, some humans are hypersensitive to horse serum proteins and may react acutely to even very small amounts of horse protein.

In view of these disadvantages, the present invention contemplates, in one embodiment, raising polyclonal antibodies to NP in birds, and in particular, in chickens. Thus, in one embodiment, the present invention contemplates a method comprising: a) providing a conserved influenza protein (e.g. NP); b) providing at least one avian species; and c) immunizing the avian species with said protein under conditions such that polyclonal antibodies to NP are produced. When birds are used, it is contemplated that the antibody will be obtained from either the bird serum or the egg. A preferred embodiment involves collection of the antibody from the egg. Laying hens export immunoglobulin to the egg yolk (“IgY”) in concentrations equal to or exceeding that found in serum. See R. Patterson et al, J. Immunol. 89:272 (1962). S. B. Carroll and B. D. Stollar, J. Biol. Chem. 258:24 (1983). In addition, the large volume of egg yolk produced vastly exceeds the volume of serum that can be safely obtained from the bird over any given time period. This is important, since administration in some embodiments of passive immunization (e.g. oral administration of unpurified antibody) can involve as much as 1-10 grams/person/day. Finally, the antibody from eggs is purer and more homogeneous; there is far less non-immunogobulin protein (as compared to serum) and only one class of immunoglobulin is transported to the yolk. This means that, in one embodiment, the yolk antibody can be processed with only simple fractionation techniques (rather than affinity purification).

It is not intended that the present invention be limited to a particular mode of immunization to generate antibodies in mammals or non-mammals; the present invention contemplates all modes of immunization, including subcutaneous, intramuscular, intraperitoneal, and intravascular injection. The present invention further contemplates immunization with or without adjuvant. (Adjuvant is defined as a substance known to increase the immune response to other antigens when administered with other antigens.) If adjuvant is used, it is not intended that the present invention be limited to any particular type of adjuvant—or that the same adjuvant, once used, be used all the time. While the present invention contemplates all types of adjuvant, whether used separately or in combinations, the preferred use of adjuvant is the use of Complete Freund's Adjuvant followed sometime later with Incomplete Freund's Adjuvant.

When immunization is used, the present invention contemplates a wide variety of immunization schedules. In one embodiment, a chicken is administered protein (e.g. NP) on day zero and subsequently receives protein in intervals thereafter. It is not intended that the present invention be limited by the particular intervals or doses. Similarly, it is not intended that the present invention be limited to any particular schedule for collecting antibody. However, a preferred schedule for immunization of the present invention is the administration of a protein (e.g. NP) on day zero at 1 mg, with subsequent administrations of the same protein at the same dose on days 14 and 21, and with gradually increasing doses (“boosts”) up to 10 mg (native protein) at approximately two week intervals up to approximately one hundred days. The preferred antibody collection time (e.g. from the eggs) is sometime after day 100.

Where birds are used and collection of antibody is performed by collecting eggs, the eggs may be stored prior to processing for antibody. It is preferred that storage of the eggs be performed at 4° C. for less than one year.

It is contemplated that chicken antibody produced in this manner can be buffer-extracted and used analytically. While unpurified, this preparation can serve as a reference for activity of the antibody prior to further manipulations (e.g., immunoaffinity purification).

When purification is used, the present invention contemplates purifying to increase the effectiveness of both non-mammalian antibody and mammalian antibody. While all types of purification (e.g., purification based on size, charge, solubility, etc.) may be used, the preferred purification approach for mammalian antibody is immunoaffinity purification. The preferred purification approaches for avian antibody are: a) Polyethylene Glycol (PEG) separation, and b) Immunoaffinity purification.

PEG purification exploits the differential solubility of lipids (which are abundant in egg yolks) and yolk proteins in high concentrations of polyethylene glycol 8000. Polson et al., Immunol. Comm. 9:495 (1980). The technique is rapid, simple, and relatively inexpensive and yields an immunoglobulin fraction that is significantly purer in terms of contaminating non-immunoglobulin proteins than the comparable ammonium sulfate fractions of mammalian sera and horse antibody. Indeed, PEG-purified antibody is sufficiently pure that the present invention contemplates, in one embodiment, the use of PEG-purified anti-NP antibody in the passive immunization of humans and animals.

Immunoaffinity purification is separation based on the affinity of antibody for specific antigen(s); antibody that binds to specific antigen(s) is separated from antibody that does not bind (under the conditions used). The present invention contemplates the use of immunoaffinity purification to dramatically reduce the foreign protein burden of anti-NP antibody by elimination of irrelevant protein (non-immunoglobulin and non-antigen-binding immunoglobulin) when the anti-NP antibody is used therapeutically. One commercially available resin for attaching NP and purifying anti-NP antibody is the aldehyde-activated resin, ACTIGEL A (available from Sterogene Bioseparations, Inc.).

Where immunoaffinity purification is used, smaller amounts of material can be administered (e.g. 0.1-2 grams/person/day, and more typically 0.05-1 grams/person/day) for passive immunization with good effect. Where administration of purified antibody is by inhalation, even smaller amount of material can be used (e.g. 0.01-0.2 grams/person/day). Administration can be for 10-14 days, but more typically, 1-5 days, and even for shorter times (e.g. 1-2 days). Longer periods of administration (e.g. 14 days to 3 months) can be employed where the exposure warrants it (e.g. in the hospital setting, in the military, etc.).
ii) Monoclonal Antibodies

In one embodiment, the present invention contemplates monoclonal antibodies specific for a conserved influenza protein, such as NP. The present invention is not limited by the methods used to generate the monoclonal antibodies or antibody fragments. Monoclonal antibodies may be made in a number of ways, including, for example, using the hybridoma method (e.g. as described by Kohler et al., Nature, 256: 495, 1975, herein incorporated by reference), or by recombinant DNA methods (e.g., U.S. Pat. No. 4,816,567, herein incorporated by reference).

Generally, in the hybridoma method, a mouse or other appropriate host animal, such as a hamster or macaque monkey, is immunized (e.g. with the immunogen such as NP from influenza) to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (e.g., Kozbor, J. Immunol., 133: 3001 (1984), herein incorporated by reference).

Culture medium in which hybridoma cells are growing is assayed for production of monoclonal antibodies directed against the antigen. Preferably, the binding specificity of monoclonal antibodies produced by hybridoma cells is determined by immuno-precipitation or by an in vitro binding assay, such as radioimmunoassay (RIA) or enzyme-linked immunoabsorbent assay (ELISA). After hybridoma cells are identified that produce antibodies of the desired specificity, affinity, and/or activity, the clones may be subcloned by limiting dilution procedures and grown by standard methods. Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography. As noted above, where purified antibody is made for passive immunization, smaller amounts of material can be administered with good effect.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies is described in more detail below.

In some embodiments, antibodies or antibody fragments are isolated from antibody phage libraries generated using the techniques described in, for example, McCafferty et al., Nature, 348: 552554 (1990). Clackson et al., Nature, 352:624-628 (1991) and Marks et al., J. Mol. Biol., 222: 581-597 (1991) describe the isolation of murine and human antibodies, respectively, using phage libraries. Subsequent publications describe the production of high affinity (nM range) human antibodies by chain shuffling (Marks et. al., BioTechnology, 10: 779-783 (1992)), as well as combinatorial infection and in vivo recombination as a strategy for constructing very large phage libraries (e.g., Waterhouse et al., Nuc. Acids. Res., 21: 2265-2266 (1993)). Thus, these techniques, and similar techniques, are viable alternatives to traditional monoclonal antibody hybridoma techniques for isolation of monoclonal antibodies.

The antibodies or antibody fragments reactive with a conserved protein of influenza (such as NP) can also be prepared, for example, by recombinant expression of immunoglobulin light and heavy chain genes in a host cell. For example, to express an antibody recombinantly, a host cell may be transfected with one or more recombinant expression vectors carrying DNA fragments encoding the immunoglobulin light and heavy chains of the antibody such that the light and heavy chains are expressed in the host cell and, preferably, secreted into the medium in which the host cell is cultured, from which medium the antibody can be recovered. Standard recombinant DNA methodologies may be used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al., all of which are herein incorporated by reference.

In certain embodiments, antibodies or antibody fragments are expressed that contain one or more of the CDRs with affinity for NP or a portion thereof. Such expression can be accomplished by first obtaining DNA fragments encoding the light and heavy chain variable regions. These DNAs can be obtained by amplification and modification of germline light and heavy chain variable sequences using the polymerase chain reaction (PCR). Germline DNA sequences for human heavy and light chain variable region genes are known in the art.

Once the germline VH and VL fragments are obtained, these sequences can be mutated to encode one or more of the CDR amino acid sequences reactive with NP. The amino acid sequences encoded by the germline VH and VL DNA sequences may be compared to the CDRs sequence(s) desired to identify amino acid residues that differ from the germline sequences. Then the appropriate nucleotides of the germline DNA sequences are mutated such that the mutated germline sequence encodes the selected CDRs, using the genetic code to determine which nucleotide changes should be made. Mutagenesis of the germline sequences may be carried out by standard methods, such as PCR-mediated mutagenesis (in which the mutated nucleotides are incorporated into the PCR primers such that the PCR product contains the mutations) or site-directed mutagenesis. In other embodiments, the variable region is synthesized de novo (e.g., using a nucleic acid synthesizer).

Once DNA fragments encoding the desired VH and VL segments are obtained (e.g., by amplification and mutagenesis of germline VH and VL genes, or synthetic synthesis, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operably linked to another DNA fragment encoding another polypeptide, such as an antibody constant region or a flexible linker. The isolated DNA encoding the VH region can be converted to a full-length heavy chain gene by operably linking the VH-encoding DNA to another DNA molecule encoding heavy chain constant regions (CH1, CH2 and CH3). The sequences of human heavy chain constant region genes are known in the art (see e.g., Kabat, E. A., et al., (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be, for example, an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operably linked to another DNA molecule encoding only the heavy chain CH1 constant region.

The isolated DNA encoding the VL region can be converted to a full-length light chain gene (as well as a Fab light chain gene) by operably linking the VL-encoding DNA to another DNA molecule encoding the light chain constant region, CL. The sequences of human light chain constant region genes are known in the art (see e.g., Kabat, E. A., et al., (1991) Sequences of Proteins of immunological Interest, Fifth Edition, U.S. Department of Health and Human Services. NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments may be operably linked to another fragment encoding a flexible linker, e.g., encoding the amino acid sequence (Gly4-Ser)3, such that the VH and VL sequences can be expressed as a contiguous single-chain protein, with the VL and VH regions joined by the flexible linker (see e.g., Huston et al., (1988) Proc. Natl. Acad. Sci. USA 85:5879-5883; and McCafferty et al., (1990) Nature 348:552-554), all of which are herein incorporated by reference).

To express the antibodies, or antibody fragments of the invention, DNAs encoding partial or full-length light and heavy chains, (e.g. obtained as described above), may be inserted into expression vectors such that the genes are operably linked to transcriptional and translational control sequences. In this context, the term “operably linked” is intended to mean that an antibody gene is ligated into a vector such that transcriptional and translational control sequences within the vector serve their intended function of regulating the transcription and translation of the antibody gene. The expression vector and expression control sequences are generally chosen to be compatible with the expression host cell used. The antibody light chain gene and the antibody heavy chain gene can be inserted into separate vectors or, more typically, both genes are inserted into the same expression vector. The antibody genes may be inserted into the expression vector by standard methods (e.g., ligation of complementary restriction sites on the antibody gene fragment and vector, or blunt end ligation if no restriction sites are present). Prior to insertion of the light or heavy chain sequences, the expression vector may already carry antibody constant region sequences. For example, one approach to converting the VH and VL sequences to full-length antibody genes is to insert them into expression vectors already encoding heavy chain constant and light chain constant regions, respectively, such that the VH segment is operably linked to the CH segment(s) within the vector and the VL segment is operably linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention may carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990), herein incorporated by reference. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma virus. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al., all of which are herein incorporated by reference.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634.665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr- host cells with methotrexate selection/amplification) and the neomycin gene (for G418 selection).

For expression of the light and heavy chains, the expression vector(s) encoding the heavy and light chains may be transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

In certain embodiments, the expression vector used to express the antibody and antibody fragments of the present invention are viral vectors, such as retro-viral vectors. Such viral vectors may be employed to generate stably transduced cell lines (e.g. for a continues source of monoclonal antibodies). In some embodiments, the GPEX gene product expression technology (from Gala Design, Inc., Middleton, Wis.) is employed to generate monoclonal antibodies. In particular embodiments, the expression technology described in WO0202783 and WO0202738 to Bleck et al. (both of which are herein incorporated by reference) is employed.

In one preferred system for recombinant expression of an antibody, or fragment thereof, a recombinant expression vector encoding both the antibody heavy chain and the antibody light chain is introduced into dhfr- CHO cells by calcium phosphate-mediated transfection. Within the recombinant expression vector, the antibody heavy and light chain genes are each operably linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector may also carry a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

In certain embodiments, the antibodies and antibody fragments of the present invention are produced in transgenic animals. For example, transgenic sheep and cows may be engineered to produce the antibodies or antibody fragments in their milk (see, e.g., Pollock D P, et al., (1999) Transgenic milk as a method for the production of recombinant antibodies. J. Immunol. Methods 231:147-157, herein incorporated by reference). The antibodies and antibody fragments of the present invention may also be produced in plants (see, e.g., Larrick et al., (2001) Production of secretory IgA antibodies in plants. Biomol. Eng. 18:87-94, herein incorporated by reference). Additional methodologies and purification protocols are provided in Humphreys et al., (2001) Therapeutic antibody production technologies: molecules applications, expression and purification, Curr. Opin. Drug Discov. Devel. 4:172-185, herein incorporated by reference. In certain embodiments, the antibodies or antibody fragments of the present invention are produced by transgenic chickens (see, e.g., US Pat. Pub. Nos. 20020108132 and 20020028488, both of which are herein incorporated by reference).

2. Administration. In one embodiment, the second step in treatment by passive immunization involves the administering of antibody to the subject. The antibodies and antibody fragments of the present invention may be administered by any suitable means, including parenteral, non-parenteral, subcutaneous, topical, intraperitoneal, intrapulmonary, intranasal, and intralesional administration. Parenteral infusions include, but are not limited to, intramuscular, intravenous, intra-arterial, intraperitoneal, or subcutaneous administration. Preferably, the dosing is given intranasally, orally or by injections, e.g. intravenous injections.

Where the antibody is raised in a non-human and administered to a human, the first concern is whether the subject will tolerate the administration of “foreign” antibody. In other words, will the subject's immune system recognize the administered antibody as antigen and mount an adverse response?

Adverse responses are typically of two types, immediate and delayed. Immediate reactions are also of two types: 1) anaphylaxis, and 2) Arthus reaction. Anaphylaxis is IgE mediated and requires sensitization to antigen. The Arthus reaction is complement dependent and requires only antibody-antigen complexes. Both immediate types of reactions are referred to as hypersensitivity reactions; the host responds as if primed by a first exposure. Such immediate reactions can be acute. Indeed, anaphylaxis, if untreated, can lead to respiratory failure and death.

Delayed reactions are caused by a host primary immune response to the foreign antibody. The reaction, called “serum sickness,” is characterized by fever, enlarged lymph glands, and joint pain. These symptoms are apparent a number of days after passive immunization and gradually subside.

The present invention further contemplates, in one embodiment, treating humans and animals by in vivo administration of antibodies which do not cause complement-associated side effects. One approach is to raise anti-NP antibodies in birds. All birds are contemplated (e.g., duck, ostrich, emu, turkey, etc.). A preferred bird is a chicken. Importantly, chicken antibody does not fix mammalian complement. See H. N. Benson et al., J. Immunol. 87:610 (1961). Thus, chicken antibody will normally not cause a complement dependent reaction. A. A. Benedict and K. Yamaga, In: Comparative Immunology (J. J. Marchaloni, Ed.), Ch. 13, Immunoglobulins and Antibody Production in Avian Species (pp. 335-375) (Blackwell, Oxford 1966).

Another approach to avoid side effects is to use human or humanized antibody. In one embodiment, humanized antibody is made (as described above) that is reactive with NP. Administration of this antibody as passive immunization (prior to or after infection with influenza) is contemplated to be beneficial with minimal side effects.

An exemplary, non-limiting range for a therapeutically or prophylactically effective amount of an antibody or antibody fragment is 0.1-20 mg/kg, more preferably 1-10 mg/kg. In some embodiments, the dosage is from 50-600 mg/m² (e.g. 375 mg/m²). It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the present invention.

The antibody and antibody fragments of the invention can be incorporated into pharmaceutical compositions suitable for administration to a subject. For example, the pharmaceutical composition may comprise an antibody or antibody fragment and a pharmaceutically acceptable carrier. As used herein, “pharmaceutically acceptable carrier” includes solvents, dispersion media, and coatings. Examples of pharmaceutically acceptable carriers include one or more of the following: water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibodies of the present invention.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies.

Sterile injectable solutions can be prepared by incorporating the active compound (i.e., antibody or antibody fragment) in the required amount in an appropriate solvent with one or a combination of ingredients enumerated above, as required, followed by sterile filtration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yield a powder of the active ingredient plus any additional desired ingredient from a previously sterile-filtered solution thereof.

3. Administration of Vectors Encoding Antibodies. While certain embodiments discussed above have involved two steps (raising antibody followed by administration), in another embodiment the present invention contemplates passive immunization by administering vector encoding antibodies to the subject. To obtain the sustained serum antibody concentration with one single injection and lower the cost of antibody protein therapy, the present invention contemplates, in one embodiment, an adenovirus-mediated full-length antibody gene therapy. In one such embodiment, full-length antibody light-chain and heavy-chain sequences are linked with internal ribosome entry site and constructed into adenoviral vector under the control of cytomegalovirus promoter. Jiang et al. Clin Cancer Res. 12:6179 (2006). An in vivo full-length antibody gene delivery system allows continuous production of a full-length antibody at high concentration after a single administration. Bioactive antibody macromolecules can be generated via gene transfer in vivo. Adenovirus-mediated antibody gene delivery can be used for the exploitation of antibodies, without being hampered by the sophisticated antibody manufacture techniques and high cost, and, furthermore, can shorten the duration and reduce the expense of antibody developments.

Of course, it is not intended that the present invention be limited to full-length antibodies. In one embodiment, an in vivo gene transfer-based therapy that uses a human adenovirus (Ad)-based vector encoding a single-chain antibody is contemplated, e.g. a single-chain antibody directed against a conserved protein such as nucleoprotein (such as influenza NP). Such a single-chain antibody has been demonstrated to be protective against other challenges. See Kasuya et al, Mol. Ther. 11:237 (2005).

In some embodiments, an unrelated sequence is inserted between the sequences encoding the heavy and light chains, such that it serves as a bridge so that these sequences can be read together. Fang et al., Nat. Biotechnol. 23:584 (2005). In one embodiment, two vectors are administered, for example, in one embodiment the present invention contemplates genetic passive immunotherapy by co-administration of Ad and AAV vectors, each encoding a NP-specific monoclonal antibody. De et al., Mol. Ther. 16:203 (2008).

Regardless of the vector design, such vectors may be administered to a variety of sites in the body. In one embodiment, a single intramuscular administration of the rAAV vector is contemplated (where the antibody molecule is synthesized and distributed to the circulatory system). Lewis et al., J. Virol. 76:8769 (2002). Alternatively, such vectors can be administered intravenously, intranasally, intrapleurally, etc. See Skaricic et al., Virology 378: 79 (2008). See Traube et al. Mol. Ther. 13:S301 (2006).

### DESCRIPTION OF PREFERRED EMBODIMENTS

It is not intended that the present invention be limited to the particular conserved protein of influenza. In one embodiment, the conserved protein is nucleoprotein (NP). It is not intended that the present invention be limited to the particular source of NP. In one embodiment, the source of NP is the NP gene of influenza A/PR8/34 (PR8) coding for the following amino acid sequence (SEQ ID NO:2):

MASQGTKRSY EQMETDGERQ NATEIRASVG KMIGGIGRFY
IQMCTELKLS DYEGRLIQNS LTIERMVLSA FDERRNKYLE
EHPSAGKDPK KTGGPIYRRV NGKWMRELIL YDKEEIRRIW
RQANNGDDAT AGLTHMMIWH SNLNDATYQR TPALVRTGMD
PRMCSLMQGS TLPRRSGAAG AAVKGVGTMV MELVRMIKRG
INDRNFWRGE NGRKTRIAYE RMCNILKGKF QTAAQKAMMD
QVRESRNPGN AEFEDLTFLA RSALILRGSV AHKSCLPACV
YGPAVASGYD FEREGYSLVG IDPFRLLQNS QVYSLIRPNE
NPAHKSQLVW MACHSAAFED LRVLSFIKGT KVLPRGKLST
RGVQIASNEN METMESSTLE LRSRYWAIRT RSGGNTNQQR
ASAGQISIQP TFSVQRNLPF DRTTIMAAFN GNTEGRTSDM
RTEIIRMMES ARPEDVSFQG RGVFELSDEK AASPIVPSFD
MSNEGSYFFG DNAEEYDN

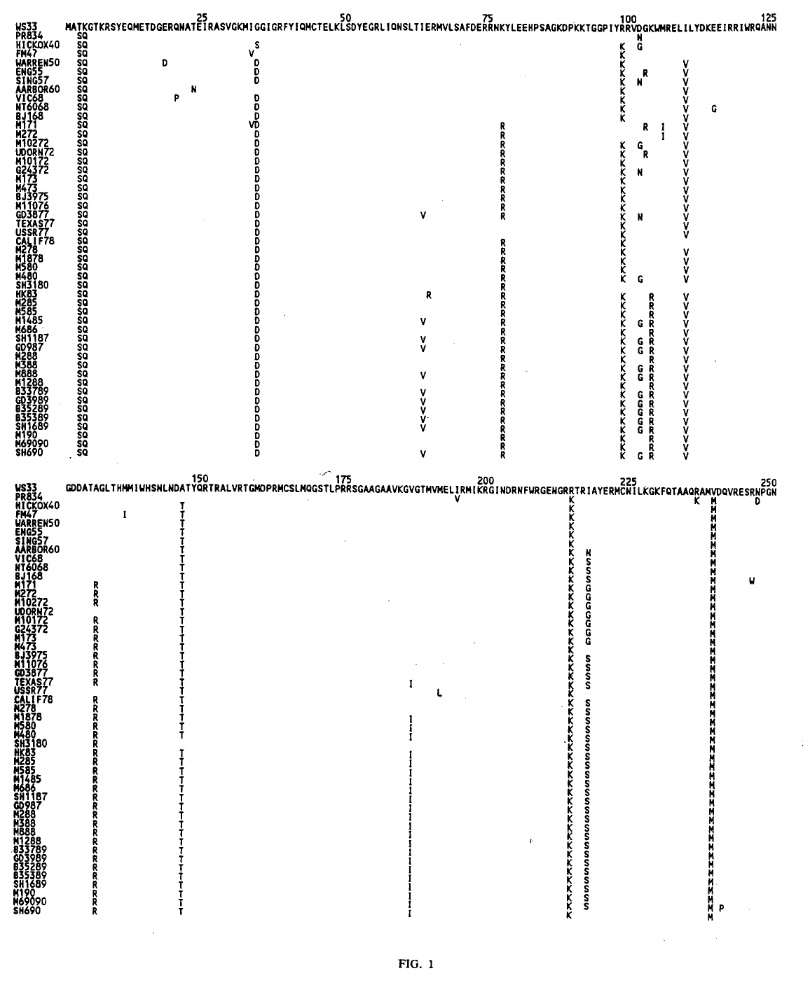

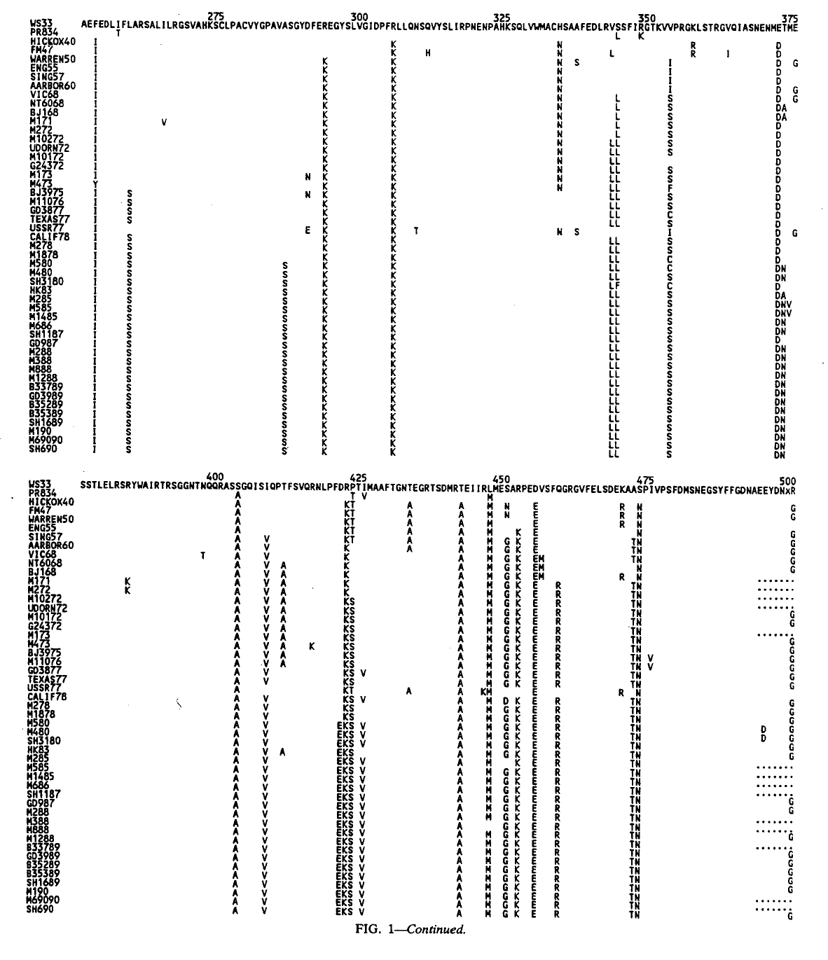

However, other sources of NP are contemplated from a variety of subtypes and strains, such as those set forth in FIG. 1 (The first identified human influenza virus, WS33, was used as a baseline; only the differences from this baseline sequence are shown).

In one particularly preferred embodiment, NP from avian influenza is contemplated. In one embodiment, the NP is from a Taiwan H5N1 strain of the Influenza A virus (A/Thailand/16/2004(H5N1)) having the amino acid sequence (SEQ ID NO: 51):

MASQGTKRSY EQMETGGERQ NATEIRASVG RMVSGIGRFY
IQMCTELKLS DYEGRLIQNS ITIERMVLSA FDERRNRYLE
EHPSAGKDPK KTGGPIYRRR DGKWVRELIL YDKEEIRRIW
RQANNGEDAT AGLTHLMIWH SNLNDATYQR TRALVRTGMD
PRMCSLMQGS TLPRRSGAAG AAVKGVGTMV MELIRMIKRG
INDRNFWRGE NGRRTRIAYE RMCNILKGKF QTAAQRAMMD
QVRESRNPGN AEIEDLIFLA RSALILRGSV AHKSCLPACV
YGLAVASGYD FEREGYSLVG IDPFRLLQNS QVFSLIRPNE
NPAHKSQLVW MACHSAAFED LRVSSFIRGT RVVPRGQLST
RGVQIASNEN MEAMDSNTLE LRSRYWAIRT RSGGNTNQQR
ASAGQISVQP TFSVQRNLPF ERATIMAAFT GNTEGRTSDM
RTEIIRMMES ARPEDVSFQG RGVFELSDEK ATNPIVPSFD
MNNEGSYFFG DNAEEYDN

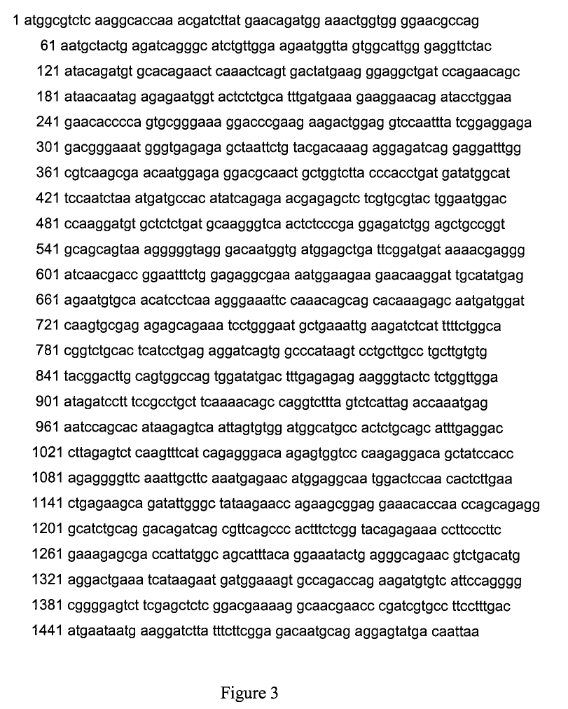

A useful nucleic acid coding sequence for the above amino acid sequence is set forth in FIG. 3 (SEQ ID NO: 52).

In yet another embodiment, the NP is from an Indonesian strain of Influenza A virus (A/Indonesia/CDC1032T/2007(H5N1) having the amino acid sequence (SEQ ID NO:53):

MASQGTKRSY EQMETGGERQ NATEIRASVG RMVSGIGRFY
IQMCTELKLS DYEGRLIQNS ITIERMVLSA FDERRNRYLE
EHPSAGKDPK KTGGPIYRRR DGKWVRELIL YDKEEIRRIW
RQANNGEDAT AGLTHLMIWH SNLNDATYQR TRALVRTGMD
PRMCSLMQGS TLPRRSGAAG AAVKGVGTMV MELIRMIKRG
INDRNFWRGE NGRRTRIAYE RMCNILKGKL QTAAQRAMMD
QVRESRNPGN AEIEDLIFLA RSALILRGSV AHKSCLPACV
YGLAVASGYD FEREGYSLVG IDPFRLLQNS QVFSLIRPNE
NPAHKSQLVW MACHSAAFED LRVSSFIRGT RVVPRGQLST
RGVQIASNEN MEVMDSNTLE LRSRYWAIRT RSGGNTNQQK
ASAGQISVQP TFSVQRNLPF ERATIMAAFT GNTEGRTSDM
RTEIIRMMES ARPEDVSFQG RGVFELSDEK ATNPIVPSFD
MNNEGSYFFG DNAEEYDN

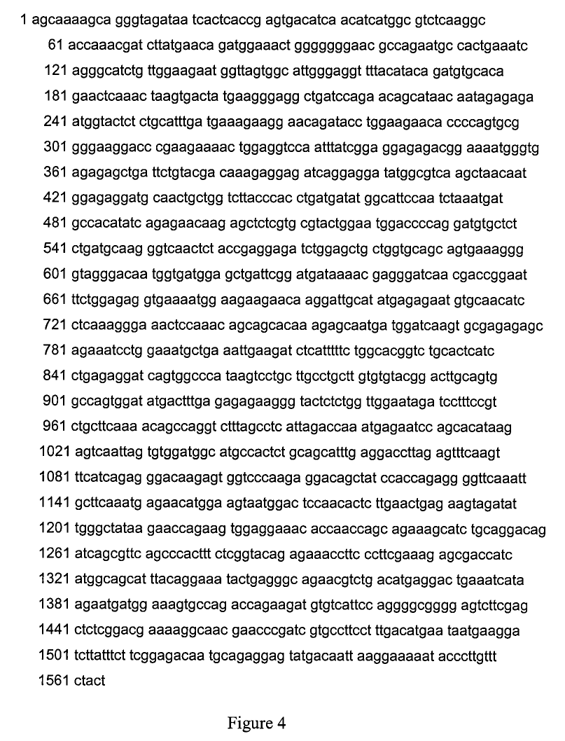

A useful nucleic acid coding sequence for the above amino acid sequence is set forth in FIG. 4 (SEQ ID NO: 54).

In yet another embodiment, the NP is from a Hong Kong strain of Influenza A virus A/Hong Kong/213/03(H5N1) having the amino acid sequence (SEQ ID NO:55):

MASQGTKRSY EQMETGGERQ NATEIRASVG RMVSGIGRFY
IQMCTELKLS DYEGRLIQNS ITIERMVLSA FDERRNRYLE
EHPSAGKDPK KTGGPIYRRR DGKWVRELIL YDKEEIRRIW
RQANNGEDAT AGLTHLMIWH SNLNDATYQR TRALVRTGMD
PRMCSLMQGS TLPRRSGAAG AAVKGVGTMV MELIRMIKRG
INDRNFWRGE NGRRTRIAYE RMCNILKGKF QTAAQRAMMD
QVRESRNPGN AEIEDLIFLA RSALILRGSV AHKSCLPACV
YGLAVASGYD FEREGYSLVG IDPFRLLQNS QVFSLIRPNE
NPAHKSQLVW MACHSAAFED LRVSSFIRGT RVVPRGQLST
RGVQIASNEN MEANDSNTLE LRSRYWAIRT RSGGNTNQQR
ASAGQISVQP TFSVQRNLPF ERSTIMAAFT GNTEGRTSDM
RTEIIRMMES ARPEDVSFQG RGVFELSDEK ATNPIVPSFD
MNNEGSYFFG DNAEEYDN

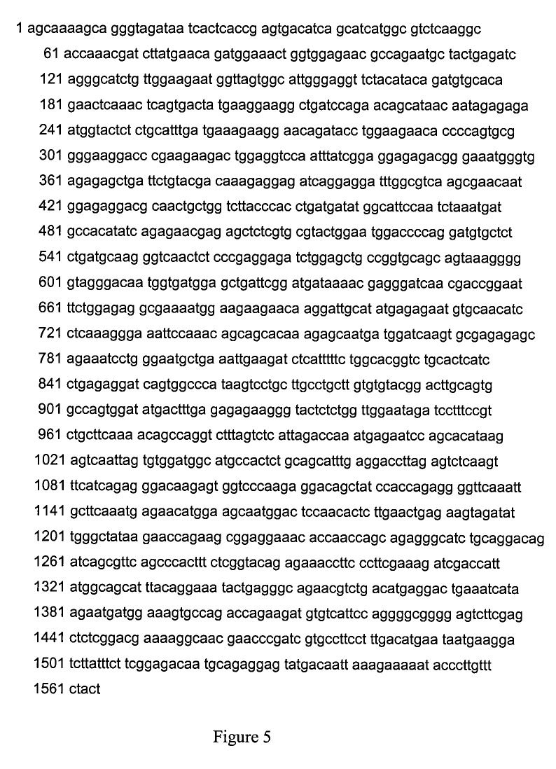

A useful nucleic acid coding sequence for the above amino acid sequence is set forth in FIG. 5 (SEQ ID NO: 56). Any of the herein described coding sequences can be used to generate large amounts of NP antigen which in turn can be used to generate large amounts of antibody (e.g. polyclonal antibody).

### Experimental

The following examples are provided in order to demonstrate and further illustrate certain preferred embodiments and aspects of the present invention and are not to be construed as limiting the scope thereof.

In the following examples, immunized mice were on the C57BL/6 background and were bred and maintained at the Trudeau Institute. B cell deficient B6.129S2-Igh-6^{tm1Cgn}/J (μMT) mice were obtained from the Jackson Laboratory. Aid^{-/-} mice lacking the capacity to isotype switch were obtained from Dr. Rachael Gerstein at the University of Massachusetts Medical School. Mice lacking the secretory exon of IgM (μS^{-/-} mice) were obtained from Dr. Ronald Corley at Boston University. Aid^{-/-} and μS^{-/-} mice were intercrossed to generate AID/μS mice, which have B cells but cannot produce antibody.

To determine viral titers in the lungs of animals, Madin-Darby Canine Kidney cells were grown in 96-well, flat-bottom plates until just confluent and then washed with HBSS. Homogenized lung samples were diluted in Zero Serum Media (Diagnostic Hybrids) supplemented with 4 μg/ml trypsin and applied to washed Madin Darby Canine Kidney cells. Plates were centrifuged for 1.5 h at 800×g, washed, and cultured overnight in Zero Serum Media/trypsin at 33° C. The medium was removed, and the cells were fixed with 80% acetone and allowed to dry. The wells were rehydrated with PBS, containing 2% FBS and 0.01% NaN₃, and probed with mouse anti-influenza A antibody (Chemicon International). The primary antibody was detected with biotinylated goat anti-mouse IgG (Chemicon International) followed by alkaline phosphatase-conjugated streptavidin (DakoCytomation). Viral foci were developed by incubating for 30 min with 5-bromo-4-chloro-3-indolyl phosphate and Nitro Blue Tetrazolium tablets (Sigma Fast BCIP/NBT from Sigma-Aldrich) dissolved in H₂O. The resulting foci were counted under a dissecting microscope. Data were analyzed for significance by Student's t test.

### EXAMPLE 1

### Production of NP Antigen

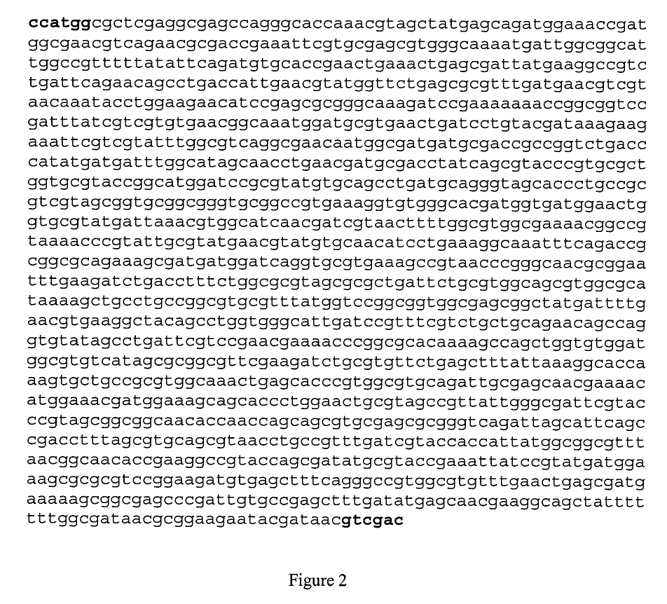

A cDNA encoding influenza nucleoprotein similar to that encoded in the A PR8/34 virus strain was designed for optimal expression in E. coli. Using the known amino acid sequence (see SEQ ID NO:2, above), the nucleotide sequence was changed for optimal codon usage in E. coli and also added restriction enzyme recognition sites at either end. Based on this deduced nucleotide sequence, cDNA was synthesized by GeneArt, with NcoI and SalI restriction endonuclease recognition sites incorporated in the synthesized product at the 5′ and 3′ ends, respectively (See FIG. 2, sequences In bold text). An alanine residue was also incorporated after the NcoI restriction site to encode a protein product in-frame with the 6× histidine tag in the expression vector. NcoI and SalI were used to digest the synthesized product as well as the pTricHis2C expression vector (Invitrogen Life Technologies), and the two products were ligated using a standard reaction. The ligation product was transformed into Top10F′ Escherichia coli (Invitrogen Life Technologies), and individual colonies were grown. Plasmid minipreps from these stocks were sequenced, and the nucleotide sequence (SEQ ID NO: 50) was as shown (FIG. 2). The amino acid sequence coded for by the nucleic acid sequence differs from the native sequence (SEQ ID NO:2) by the fact that the beginning amino acids are: MALEASQ . . . etc.; and the end contains the his tag.

Upon confirming the correct nucleotide sequence, the transformed bacteria were grown to exponential phase and protein expression was induced with isopropyl-β-D-thiogalactopyranoside. Cells were lysed by sonication in hypertonic buffer. The recombinant protein was purified using the ProBond Purification system from Invitrogen Life Technologies. Purified recombinant protein was dialyzed against phosphate-buffered saline and sterile-filtered before use.

### EXAMPLE 2

### Active Immunization With NP

Mice were immunized with 30 μg recombinant nucleoprotein (rNP) in combination with 20 μg lipopolysaccharide on days 0 and 10 by intraperitoneal injection. An equal number of control mice were injected with LPS alone.

Serum was obtained 39 days after initial immunization to measure anti-NP antibody by ELISA. Briefly, peripheral blood was obtained from either euthanized mice by severing the renal artery and pipetting into a 1.5-ml tube or from live mice via the lateral tail vein. After clotting for 30 min at 37° C., the precipitate was pelleted in a microcentrifage, and the serum was collected. NP-specific ELISAs were performed by coating plates with 2 μg/ml rNP. Serum samples were diluted in 3-fold serial dilutions in PBS with 10 μg/ml BSA and 0.1% Tween 20 before incubation on coated plates. Bound antibody was detected with HRP-conjugated goat anti-mouse IgM or goat anti-mouse IgG (Southern Biotechnology Associates).

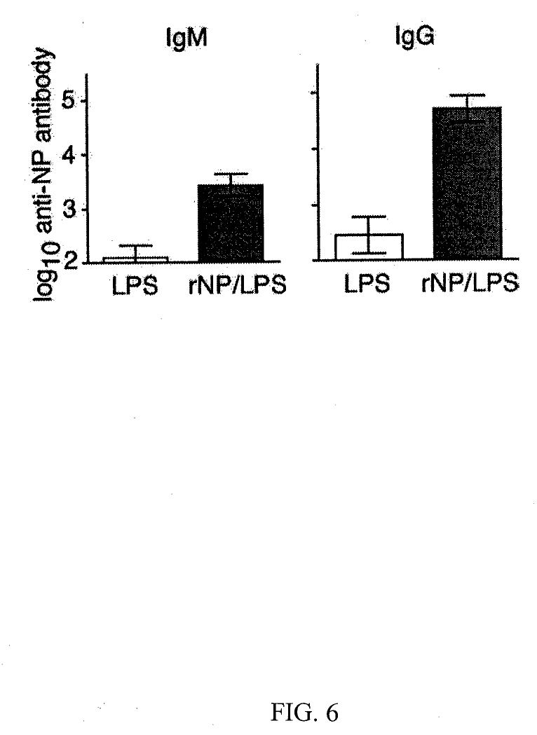

This immunization schedule alone did not induce an NP-specific CD8 T cell response that was detectable by MHC class I tetramer staining and flow cytometry at various times after boosting (data not shown). However, the immunization clearly induced high titers of NP-specific antibody in the serum as quickly as 39 days after priming (FIG. 6 shows mean plus/minus SD of five mice per group). Thus, immunization with soluble rNP promotes a robust antibody response, but a limited CD8 T cell response.

### EXAMPLE 3

### Protection After Active Immunization

In this example, in order to determine whether this NP-based vaccine could confer protection from an influenza virus challenge, the immunized mice were anaesthetized with isofluorane USP (Webster Veterinary) and intranasally (i.n.) infected with a non-lethal dose of influenza PR8 virus (500 EIU, ˜0.2 LD₅₀) in 100 ml sterile PBS one month after the boost (day 40 after priming).

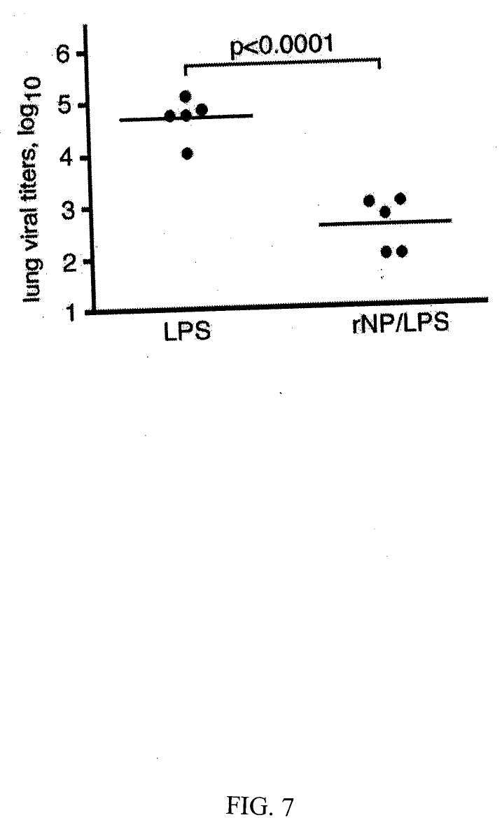

Mice immunized with LPS alone lost ˜15% body weight by day 7 post-infection, and had not yet recovered to their initial starting weight by day 11; by contrast, mice vaccinated with rNP/LPS lost less than 5% of their initial weight, and fully recovered by day 11 (data not shown). The reduced morbidity in rNP-vaccinated mice was associated with significantly lower viral titers in the lungs on day 8 after infection (FIG. 7). Therefore, as previously described, immunization of C57BL/6 mice with rNP provides some measure of protection from sublethal challenge. Tamura et al. J. Immunol. 156:3892-3900 (1996). Cox et al, Scand. J. Immunol. 55:14-23 (2002).

### EXAMPLE 4

### Protection After Passive Immunization

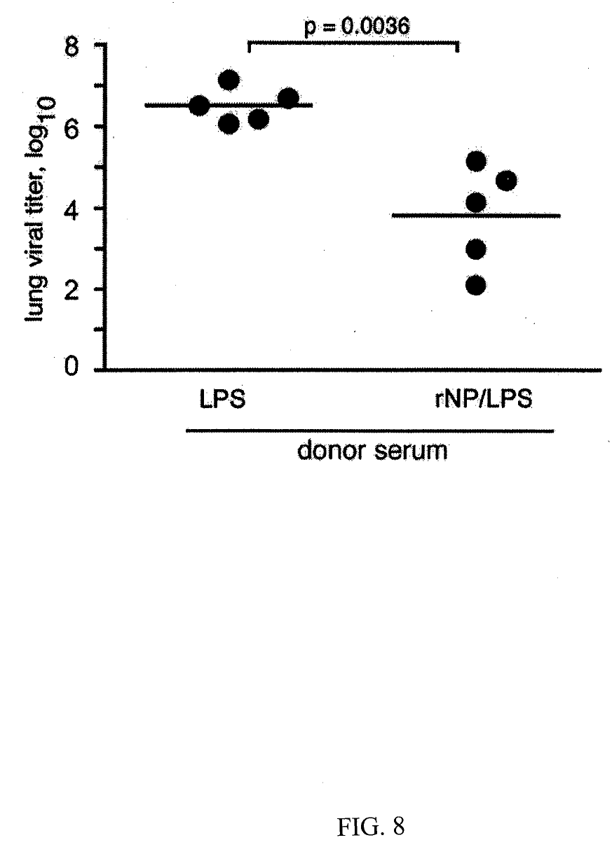

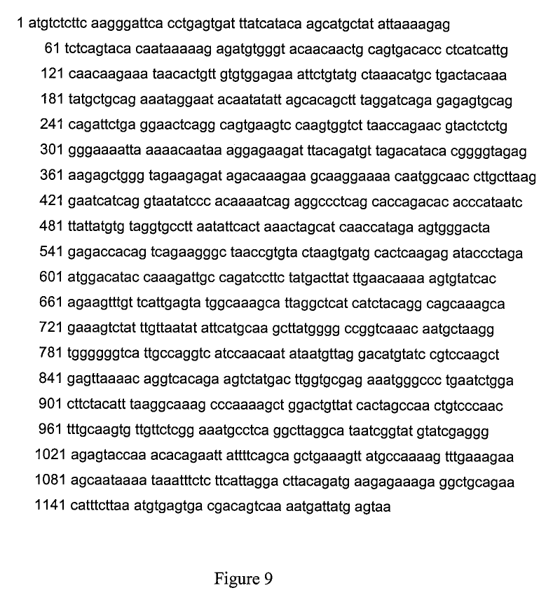

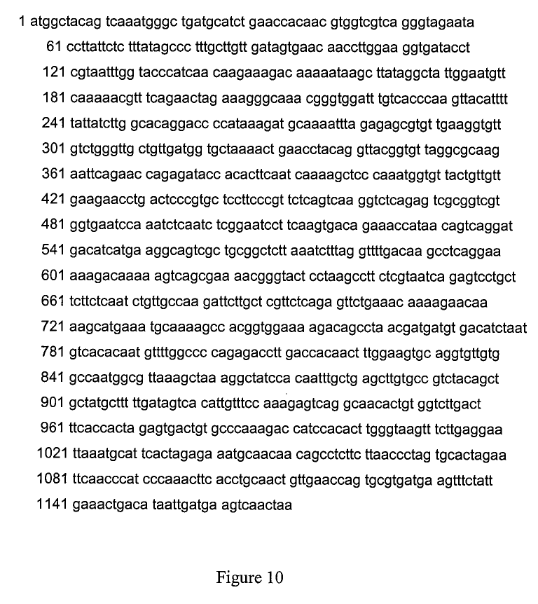

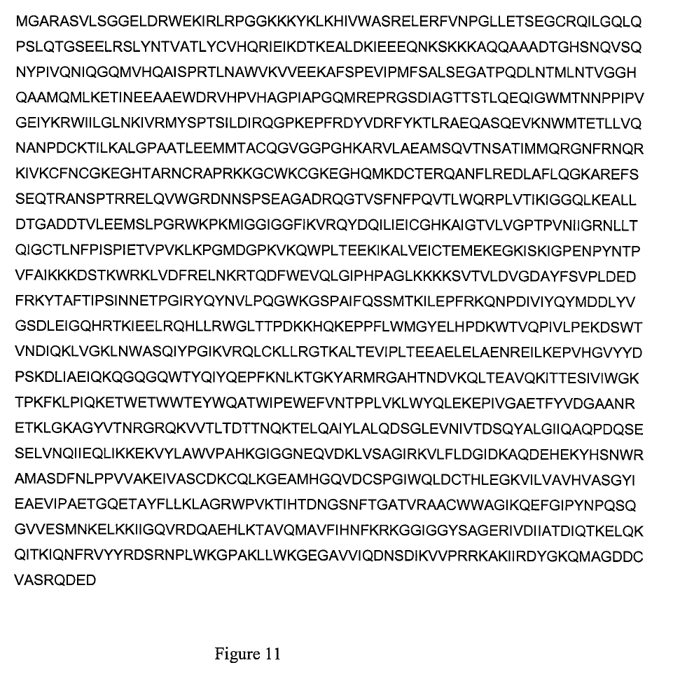

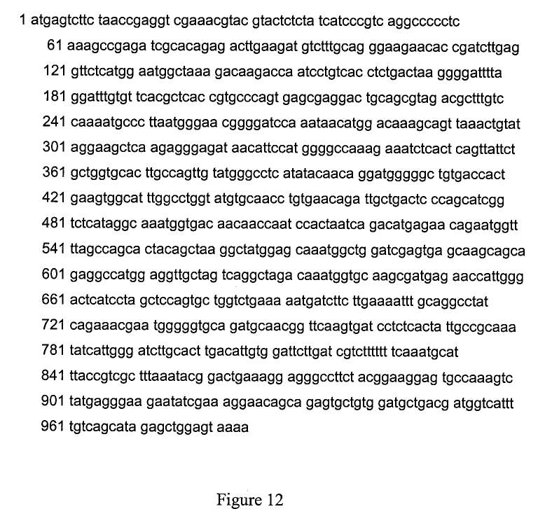

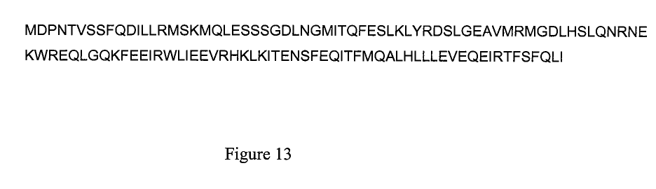

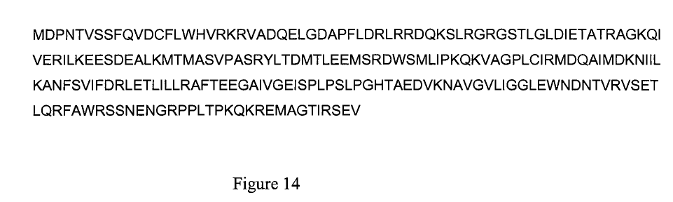

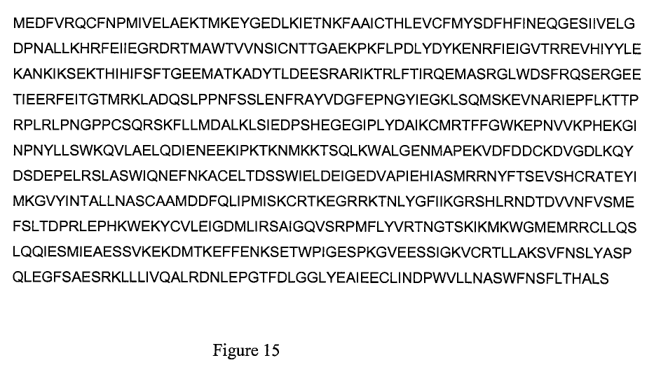

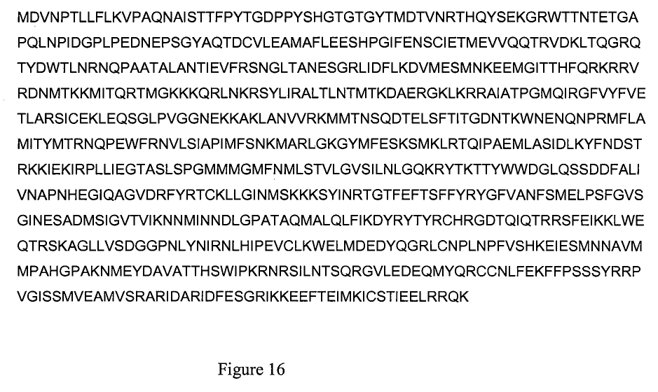

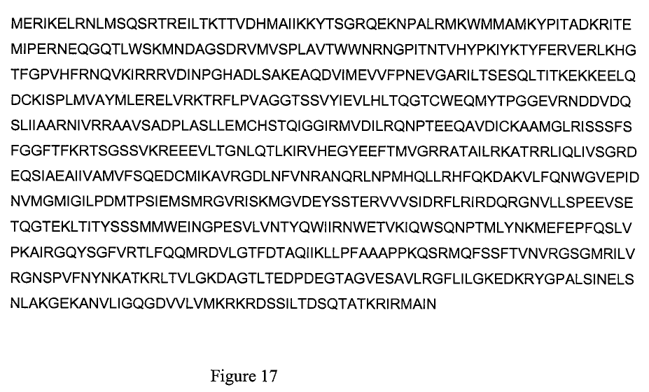

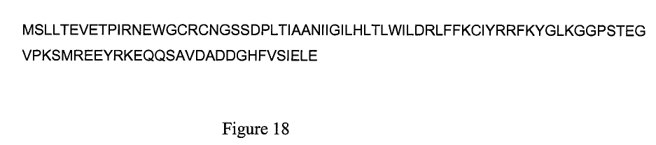

In this example, serum from rNP-vaccinated C57BL/6 donors was transferred to μMT mice, and B cell-deficient recipients were challenged the with influenza virus the following day. Whereas recipients of LPS-immune serum (the control) continued to lose up to 25% of initial body weight through day 10 after infection, μMT mice receiving rNP-immune serum lost only about 10% of their body weight, and began to recover by day 8 (data not shown). Moreover, lung viral titers on day 10 were reduced by ˜100-fold in recipients of rNP-immune serum relative to those in mice that received control serum (FIG. 8). Therefore, rNP-immune serum can convey protection against influenza challenge in T cell-competent μMT hosts.

To demonstrate that the protection conveyed by rNP-immune serum transfer is antibody-mediated, we immunized C57BL/6 and antibody-deficient AID/□S mice with rNP/LPS, transferred serum from these animals to naïve □MT recipients, and challenged them with influenza virus the following day. Recipients of rNP-immune serum from C57BL/6 mice lost only about 15% of their initial body weight, and were recovering by day 11 post-infection; however, mice that received serum from rNP-immune AID/□S mice still lost >25% body weight and showed no recovery—effects comparable to recipients of C57BL/6 control serum (LPS) (data not shown). Additionally, rNP-immune serum from the AID/μS donors failed to reduce lung viral titers (data not shown). While not intended to limit the invention to any particular mechanism, these results suggest that the protection against influenza infection conveyed by rNP-immune serum transfer is dependent upon antibody.

To address why previous studies found no protective effect of NP-specific antibodies in scid mice, rNP-immune serum was transferred into mice deficient in recombination-activating gene 1 (Rag1^{-/-}), which, similar to scid mice, lack both B cells and T cells due to a requirement for this enzyme during lymphopoiesis. In contrast to μMT recipients, which lack mature B cells, but have T cells, Rag1^{-/-} mice that received rNP-immune serum had the same amount of virus in the lung on day 10 compared with mice receiving control serum (data not shown). While not intended to limit the invention to any particular mechanism, these results suggest that T lymphocytes are required for immune protection conferred by NP-immune antibody.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention are intended to be within the scope of the following claims.

1. A method for treating a viral infection comprising:
a) providing:
i) a subject exhibiting symptoms of a viral infection,
ii) a composition comprising antibody or fragment thereof reactive with a viral nucleoprotein;
b) administering said composition to said subject under conditions such that said symptoms are reduced.

2. The method of claim 1, wherein said viral infection is infection by Influenza A.

3. The method of claim 1, wherein said subject is a bird.

4. The method of claim 1, wherein said subject is a mammal.

5. The method of claim 4, wherein said mammal is a human.

6. The method of claim 5, wherein said human is a child.

7. The method of claim 4, wherein said human is elderly.

8. The method of claim 4, wherein said human is immunocompromised.

9. The method of claim 1, wherein said antibody is polyclonal.

10. The method of claim 1, wherein said antibody is monoclonal.

11. The method of claim 9, wherein said antibody was raised in a non-human mammal immunized with influenza viral nucleoprotein.

12. The method of claim 9, wherein said antibody was raised in a non-mammal immunized with influenza viral nucleoprotein.

13. The method of claim 12, wherein said non-mammal is a bird.

14. A method for treating an Influenza A viral infection comprising:
a) providing:
i) a subject exhibiting symptoms of an Influenza A viral infection,
ii) a composition comprising antibody or a fragment thereof reactive with Influenza A viral protein selected from the group consisting of PB1, PB2, PA, NP, NS1, NS2, M1, M2 and PB1-F2;
b) administering said composition to said subject under conditions such that said symptoms are reduced.

15. The method of claim 14, wherein said Influenza A is of avian origin.

16. The method of claim 14, wherein said Influenza A is of swine origin.

17. The method of claim 14, wherein said Influenza A is of equine origin.

18. The method of claim 14, wherein said subject is a bird.

19. The method of claim 14, wherein said subject is a mammal.

20. The method of claim 19, wherein said mammal is a human.

21. The method of claim 20, wherein said human is a child.

22. The method of claim 20, wherein said human is elderly.

23. The method of claim 20, wherein said human is immunocompromised.

24. The method of claim 14, wherein said antibody is polyclonal.

25. The method of claim 14, wherein said antibody is monoclonal.

26. The method of claim 24, wherein said antibody was raised in a non-human mammal immunized with Influenza A viral nucleoprotein.

27. The method of claim 24, wherein said antibody was raised in a non-mammal immunized with Influenza A viral nucleoprotein.

28. The method of claim 27, wherein said non-mammal is a bird.

29. A method for protecting against an Influenza A viral infection comprising:
a) providing:
i) a subject at risk for an Influenza A viral infection,
ii) a composition comprising antibody or fragment thereof reactive with Influenza A viral protein selected from the group consisting of PB1, PB2, PA, NP, NS1, NS2, M1, M2 and PB1-F2;
b) administering said composition to said subject prior to any symptoms of infection.

30. The method of claim 29, wherein said Influenza A is of avian origin.

31. The method of claim 29, wherein said Influenza A is of swine origin.

32. The method of claim 29, wherein said Influenza A is of equine origin.

33. The method of claim 29, wherein said subject is a non-mammal.

34. The method of claim 33, wherein said non-mammal is a bird.

35. The method of claim 29, wherein said subject is a mammal.

36. The method of claim 35, wherein said mammal is a human.

37. The method of claim 36, wherein said human is a child.

38. The method of claim 36, wherein said human is elderly.

39. The method of claim 36, wherein said human is immunocompromised.

40. The method of claim 29, wherein said antibody is polyclonal.

41. The method of claim 29, wherein said antibody is monoclonal.

42. The method of claim 40, wherein said antibody was raised in a non-human mammal immunized with Influenza A viral nucleoprotein.

43. The method of claim 40, wherein said antibody was raised in a non-mammal immunized with Influenza A viral nucleoprotein.

44. The method of claim 43, wherein said non-mammal is a bird.

45. A method for treating a viral infection comprising:
a) providing:
i) a subject exhibiting symptoms of a viral infection,
ii) a composition comprising a vector encoding antibody or an antibody fragment reactive with a viral nucleoprotein;
b) administering said composition to said subject under conditions such that antibody is produced in said subject and said symptoms are reduced.

46. The method of claim 45, wherein said vector comprises antibody light-chain and heavy-chain nucleic acid sequences linked with an internal ribosome entry site and constructed into an adenoviral vector under the control of a promoter.


SEQUENCE LISTING (68 sequences)

SEQ ID NO: 1 - protein, 500 aa, Influenza A virus
  misc_feature (499)..(499): Xaa can be any naturally occurring amino acid
MATKGTKRSYEQMETDGERQNATEIRASVGKMIGGIGRFYIQMCTELKLSDYEGRLIQNSLTIERMVLSAFDERRNKYLEEHPSAGKDPKKTGGPIYRRVDGKWMRELILYDKEEIRRIWRQANNGDDATAGLTHMMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMVDQVRESRNPGNAEFEDLIFLARSALILRGSVAHKSCLPACVYGPAVASGYDFEREGYSLVGIDPFRLLQNSQVYSLIRPNENPAHKSQLVWMACHSAAFEDLRVSSFIRGTKVVPRGKLSTRGVQIASNENMETMESSTLELRSRYWAIRTRSGGNTNQQRASSGQISIQPTFSVQRNLPFDRPTIMAAFTGNTEGRTSDMRTEIIRLMESARPEDVSFQGRGVFELSDEKAASPIVPSFDMSNEGSYFFGDNAEEYDNXR

SEQ ID NO: 2 - protein, 498 aa, Influenza A virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 3 - protein, 24 aa, Influenza A virus
SQSKGTKMIKNRDAKTAAMNERNG

SEQ ID NO: 4 - protein, 26 aa, Influenza A virus
SQVKITKMIKHNLRDAKTAAMNERNG

SEQ ID NO: 5 - protein, 26 aa, Influenza A virus
SQDDKVTKMIKKNSIDGAKTAAMERN

SEQ ID NO: 6 - protein, 25 aa, Influenza A virus
SQDKRVTKMIKKNIDAKTAAMKENG

SEQ ID NO: 7 - protein, 28 aa, Influenza A virus
SQDKNVTKMIKKNIDAVKTAAMGKETNG

SEQ ID NO: 8 - protein, 28 aa, Influenza A virus
SQNKVTKNMIKKNIDGAVKAAMGKETNG

SEQ ID NO: 9 - protein, 31 aa, Influenza A virus
SQPDKVTKSMIKKNLSDGTAVKAMGKEMTNG

SEQ ID NO: 10 - protein, 30 aa, Influenza A virus
SQDKVGTKSMIKKNLSDAAVAKAMGKEMNG

SEQ ID NO: 11 - protein, 30 aa, Influenza A virus
SQDKVTKSMWIKKNLSDAAVAKAMGKEMRN

SEQ ID NO: 12 - protein, 34 aa, Influenza A virus
SQVDRRIVRTKGMIVKKNLSDKAVAKAMGKERTN

SEQ ID NO: 13 - protein, 31 aa, Influenza A virus
SQDRIVRTKGMIKKNLSDKAVAKAMGKERTN

SEQ ID NO: 14 - protein, 33 aa, Influenza A virus
SQDRKGVRTKGMIKKNLLSDAVAKSAMGKERTN

SEQ ID NO: 15 - protein, 33 aa, Influenza A virus
SQDRKRVTKGMIKKNLLSDAVAKSAMGKERTNG

SEQ ID NO: 16 - protein, 32 aa, Influenza A virus
SQDRKVRTKGMIKKNLLDAVAKSAMGKERTNG

SEQ ID NO: 17 - protein, 33 aa, Influenza A virus
SQDRKNVRTKGMIKKNLLSDAVAKSAMGKERTN

SEQ ID NO: 18 - protein, 34 aa, Influenza A virus
SQDRKVRTKGMINKKNLLSDAVAKSAMGKERTNG

SEQ ID NO: 19 - protein, 33 aa, Influenza A virus
SQDRKVRTKMYKKNLLFDAVAKKSAMGKERTNG

SEQ ID NO: 20 - protein, 35 aa, Influenza A virus
SQDRKVRTKSMISNKKLLSDAVAKSAMGKERTNVG

SEQ ID NO: 21 - protein, 34 aa, Influenza A virus
SQDRKVRTKSMISKKLLSDAVAKSAMGKERTNVG

SEQ ID NO: 22 - protein, 35 aa, Influenza A virus
SQDVRKNVRTKSMISKKLLCDAVKSVAMGKERTNG

SEQ ID NO: 23 - protein, 32 aa, Influenza A virus
SQDKVRTIKSMISKKLLSDAVKSAMGKERTNG

SEQ ID NO: 24 - protein, 28 aa, Influenza A virus
SQDKVTLKMIEKKTNSIDGAKTAAKMRN

SEQ ID NO: 25 - protein, 32 aa, Influenza A virus
SQDRKRTKSMISKKLLSDAVKSVAMDKERTNG

SEQ ID NO: 26 - protein, 32 aa, Influenza A virus
SQDRKVRTKSMISKKLLSDAVKSAMGKERTNG

SEQ ID NO: 27 - protein, 33 aa, Influenza A virus
SQDRKVRTIKSMISKKLLCDAVKSAMGKERTNG

SEQ ID NO: 28 - protein, 38 aa, Influenza A virus
SQDRKVRTIKSMISSKKLLCDNAVEKSVAMGKERTNDG

SEQ ID NO: 29 - protein, 39 aa, Influenza A virus
SQDRKGVRTIKSMISSKKLLSDNAVEKSVAMGKERTNDG

SEQ ID NO: 30 - protein, 32 aa, Influenza A virus
SQDRRKSMISSKKLFCDAVEKSVAMGKERTNG

SEQ ID NO: 31 - protein, 39 aa, Influenza A virus
SQDRRKRVRTIKSMISSKKLLSDAAVAEKSAMGKERTNG

SEQ ID NO: 32 - protein, 37 aa, Influenza A virus
SQDRKRVRTIKSMISSKKLLSDNVAVEKSVAMKERTN

SEQ ID NO: 33 - protein, 38 aa, Influenza A virus
SQDRKRVRTIKSMISSKKLLSDNVAVEKSVAMGKERTN

SEQ ID NO: 34 - protein, 39 aa, Influenza A virus
SQDVRKGRVRTIKSMISSKKLLSDNAVEKSVAMGKERTN

SEQ ID NO: 35 - protein, 37 aa, Influenza A virus
SQDRKRVRTIKSMISSKKLLSDNAVEKSVAMGKERTN

SEQ ID NO: 36 - protein, 39 aa, Influenza A virus
SQDVRKGRVRTIKSMISSKKLLSDAVEKSVAMGKERTNG

SEQ ID NO: 37 - protein, 40 aa, Influenza A virus
SQDVRKGRVRTIKSMISSKKLLSDNAVEKSVAMGKERTNG

SEQ ID NO: 38 - protein, 37 aa, Influenza A virus; identical to SEQ ID NO: 35

SEQ ID NO: 39 - protein, 37 aa, Influenza A virus
SQDRKGRVRTIKSMISSKKLLSDNAVEKSVAGKERTN

SEQ ID NO: 40 - protein, 40 aa, Influenza A virus; identical to SEQ ID NO: 37

SEQ ID NO: 41 - protein, 37 aa, Influenza A virus; identical to SEQ ID NO: 35

SEQ ID NO: 42 - protein, 40 aa, Influenza A virus; identical to SEQ ID NO: 37

SEQ ID NO: 43 - protein, 40 aa, Influenza A virus; identical to SEQ ID NO: 37

SEQ ID NO: 44 - protein, 40 aa, Influenza A virus; identical to SEQ ID NO: 37

SEQ ID NO: 45 - protein, 30 aa, Influenza A virus
SQDVRKGRVRTIKSMAVEKSVAMGKERTNG

SEQ ID NO: 46 - protein, 30 aa, Influenza A virus; identical to SEQ ID NO: 45

SEQ ID NO: 47 - protein, 27 aa, Influenza A virus
SQDRKRVRTIKSMAVEKSVAMGKERTN

SEQ ID NO: 48 - protein, 28 aa, Influenza A virus
SQDRKRVRTIKSMPAVEKSVAMGKERTN

SEQ ID NO: 49 - protein, 29 aa, Influenza A virus
SQDVRKGRVRTIKMAVEKSVAMGKERTNG

SEQ ID NO: 50 - DNA, 1511 bp, Influenza A virus
ccatggcgctcgaggcgagccagggcaccaaacgtagctatgagcagatggaaaccgatggcgaacgtcagaacgcgaccgaaattcgtgcgagcgtgggcaaaatgattggcggcattggccgtttttatattcagatgtgcaccgaactgaaactgagcgattatgaaggccgtctgattcagaacagcctgaccattgaacgtatggttctgagcgcgtttgatgaacgtcgtaacaaatacctggaagaacatccgagcgcgggcaaagatccgaaaaaaaccggcggtccgatttatcgtcgtgtgaacggcaaatggatgcgtgaactgatcctgtacgataaagaagaaattcgtcgtatttggcgtcaggcgaacaatggcgatgatgcgaccgccggtctgacccatatgatgatttggcatagcaacctgaacgatgcgacctatcagcgtacccgtgcgctggtgcgtaccggcatggatccgcgtatgtgcagcctgatgcagggtagcaccctgccgcgtcgtagcggtgcggcgggtgcggccgtgaaaggtgtgggcacgatggtgatggaactggtgcgtatgattaaacgtggcatcaacgatcgtaacttttggcgtggcgaaaacggccgtaaaacccgtattgcgtatgaacgtatgtgcaacatcctgaaaggcaaatttcagaccgcggcgcagaaagcgatgatggatcaggtgcgtgaaagccgtaacccgggcaacgcggaatttgaagatctgacctttctggcgcgtagcgcgctgattctgcgtggcagcgtggcgcataaaagctgcctgccggcgtgcgtttatggtccggcggtggcgagcggctatgattttgaacgtgaaggctacagcctggtgggcattgatccgtttcgtctgctgcagaacagccaggtgtatagcctgattcgtccgaacgaaaacccggcgcacaaaagccagctggtgtggatggcgtgtcatagcgcggcgttcgaagatctgcgtgttctgagctttattaaaggcaccaaagtgctgccgcgtggcaaactgagcacccgtggcgtgcagattgcgagcaacgaaaacatggaaacgatggaaagcagcaccctggaactgcgtagccgttattgggcgattcgtacccgtagcggcggcaacaccaaccagcagcgtgcgagcgcgggtcagattagcattcagccgacctttagcgtgcagcgtaacctgccgtttgatcgtaccaccattatggcggcgtttaacggcaacaccgaaggccgtaccagcgatatgcgtaccgaaattatccgtatgatggaaagcgcgcgtccggaagatgtgagctttcagggccgtggcgtgtttgaactgagcgatgaaaaagcggcgagcccgattgtgccgagctttgatatgagcaacgaaggcagctatttttttggcgataacgcggaagaatacgataacgtcgac

SEQ ID NO: 51 - protein, 498 aa, Influenza A virus
MASQGTKRSYEQMETGGERQNATEIRASVGRMVSGIGRFYIQMCTELKLSDYEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMEAMDSNTLELRSRYWAIRTRSGGNTNQQRASAGQISVQPTFSVQRNLPFERATIMAAFTGNTEGRTSDMRTEIIRMMESARPEDVSFQGRGVFELSDEKATNPIVPSFDMNNEGSYFFGDNAEEYDN

SEQ ID NO: 52 - DNA, 1497 bp, Influenza A virus
atggcgtctcaaggcaccaaacgatcttatgaacagatggaaactggtggggaacgccagaatgctactgagatcagggcatctgttggaagaatggttagtggcattgggaggttctacatacagatgtgcacagaactcaaactcagtgactatgaagggaggctgatccagaacagcataacaatagagagaatggtactctctgcatttgatgaaagaaggaacagatacctggaagaacaccccagtgcgggaaaggacccgaagaagactggaggtccaatttatcggaggagagacgggaaatgggtgagagagctaattctgtacgacaaagaggagatcaggaggatttggcgtcaagcgaacaatggagaggacgcaactgctggtcttacccacctgatgatatggcattccaatctaaatgatgccacatatcagagaacgagagctctcgtgcgtactggaatggacccaaggatgtgctctctgatgcaagggtcaactctcccgaggagatctggagctgccggtgcagcagtaaagggggtagggacaatggtgatggagctgattcggatgataaaacgagggatcaacgaccggaatttctggagaggcgaaaatggaagaagaacaaggattgcatatgagagaatgtgcaacatcctcaaagggaaattccaaacagcagcacaaagagcaatgatggatcaagtgcgagagagcagaaatcctgggaatgctgaaattgaagatctcatttttctggcacggtctgcactcatcctgagaggatcagtggcccataagtcctgcttgcctgcttgtgtgtacggacttgcagtggccagtggatatgactttgagagagaagggtactctctggttggaatagatcctttccgcctgcttcaaaacagccaggtctttagtctcattagaccaaatgagaatccagcacataagagtcaattagtgtggatggcatgccactctgcagcatttgaggaccttagagtctcaagtttcatcagagggacaagagtggtcccaagaggacagctatccaccagaggggttcaaattgcttcaaatgagaacatggaggcaatggactccaacactcttgaactgagaagcagatattgggctataagaaccagaagcggaggaaacaccaaccagcagagggcatctgcaggacagatcagcgttcagcccactttctcggtacagagaaaccttcccttcgaaagagcgaccattatggcagcatttacaggaaatactgagggcagaacgtctgacatgaggactgaaatcataagaatgatggaaagtgccagaccagaagatgtgtcattccaggggcggggagtcttcgagctctcggacgaaaaggcaacgaacccgatcgtgccttcctttgacatgaataatgaaggatcttatttcttcggagacaatgcagaggagtatgacaattaa

SEQ ID NO: 53 - protein, 498 aa, Influenza A virus
MASQGTKRSYEQMETGGERQNATEIRASVGRMVSGIGRFYIQMCTELKLSDYEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKLQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMEVMDSNTLELRSRYWAIRTRSGGNTNQQKASAGQISVQPTFSVQRNLPFERATIMAAFTGNTEGRTSDMRTEIIRMMESARPEDVSFQGRGVFELSDEKATNPIVPSFDMNNEGSYFFGDNAEEYDN

SEQ ID NO: 54 - DNA, 1565 bp, Influenza A virus
agcaaaagcagggtagataatcactcaccgagtgacatcaacatcatggcgtctcaaggcaccaaacgatcttatgaacagatggaaactgggggggaacgccagaatgccactgaaatcagggcatctgttggaagaatggttagtggcattgggaggttttacatacagatgtgcacagaactcaaactaagtgactatgaagggaggctgatccagaacagcataacaatagagagaatggtactctctgcatttgatgaaagaaggaacagatacctggaagaacaccccagtgcggggaaggacccgaagaaaactggaggtccaatttatcggaggagagacggaaaatgggtgagagagctgattctgtacgacaaagaggagatcaggaggatatggcgtcaagctaacaatggagaggatgcaactgctggtcttacccacctgatgatatggcattccaatctaaatgatgccacatatcagagaacaagagctctcgtgcgtactggaatggaccccaggatgtgctctctgatgcaagggtcaactctaccgaggagatctggagctgctggtgcagcagtgaaaggggtagggacaatggtgatggagctgattcggatgataaaacgagggatcaacgaccggaatttctggagaggtgaaaatggaagaagaacaaggattgcatatgagagaatgtgcaacatcctcaaagggaaactccaaacagcagcacaaagagcaatgatggatcaagtgcgagagagcagaaatcctggaaatgctgaaattgaagatctcatttttctggcacggtctgcactcatcctgagaggatcagtggcccataagtcctgcttgcctgcttgtgtgtacggacttgcagtggccagtggatatgactttgagagagaagggtactctctggttggaatagatcctttccgtctgcttcaaaacagccaggtctttagcctcattagaccaaatgagaatccagcacataagagtcaattagtgtggatggcatgccactctgcagcatttgaggaccttagagtttcaagtttcatcagagggacaagagtggtcccaagaggacagctatccaccagaggggttcaaattgcttcaaatgagaacatggaagtaatggactccaacactcttgaactgagaagtagatattgggctataagaaccagaagtggaggaaacaccaaccagcagaaagcatctgcaggacagatcagcgttcagcccactttctcggtacagagaaaccttcccttcgaaagagcgaccatcatggcagcatttacaggaaatactgagggcagaacgtctgacatgaggactgaaatcataagaatgatggaaagtgccagaccagaagatgtgtcattccaggggcggggagtcttcgagctctcggacgaaaaggcaacgaacccgatcgtgccttcctttgacatgaataatgaaggatcttatttcttcggagacaatgcagaggagtatgacaattaaggaaaaatacccttgtttctact

SEQ ID NO: 55 - protein, 498 aa, Influenza A virus
MASQGTKRSYEQMETGGERQNATEIRASVGRMVSGIGRFYIQMCTELKLSDYEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMEAMDSNTLELRSRYWAIRTRSGGNTNQQRASAGQISVQPTFSVQRNLPFERSTIMAAFTGNTEGRTSDMRTEIIRMMESARPEDVSFQGRGVFELSDEKATNPIVPSFDMNNEGSYFFGDNAEEYDN

SEQ ID NO: 56 - DNA, 1565 bp, Influenza A virus
agcaaaagcagggtagataatcactcaccgagtgacatcagcatcatggcgtctcaaggcaccaaacgatcttatgaacagatggaaactggtggagaacgccagaatgctactgagatcagggcatctgttggaagaatggttagtggcattgggaggttctacatacagatgtgcacagaactcaaactcagtgactatgaaggaaggctgatccagaacagcataacaatagagagaatggtactctctgcatttgatgaaagaaggaacagatacctggaagaacaccccagtgcggggaaggacccgaagaagactggaggtccaatttatcggaggagagacgggaaatgggtgagagagctgattctgtacgacaaagaggagatcaggaggatttggcgtcaagcgaacaatggagaggacgcaactgctggtcttacccacctgatgatatggcattccaatctaaatgatgccacatatcagagaacgagagctctcgtgcgtactggaatggaccccaggatgtgctctctgatgcaagggtcaactctcccgaggagatctggagctgccggtgcagcagtaaagggggtagggacaatggtgatggagctgattcggatgataaaacgagggatcaacgaccggaatttctggagaggcgaaaatggaagaagaacaaggattgcatatgagagaatgtgcaacatcctcaaagggaaattccaaacagcagcacaaagagcaatgatggatcaagtgcgagagagcagaaatcctgggaatgctgaaattgaagatctcatttttctggcacggtctgcactcatcctgagaggatcagtggcccataagtcctgcttgcctgcttgtgtgtacggacttgcagtggccagtggatatgactttgagagagaagggtactctctggttggaatagatcctttccgtctgcttcaaaacagccaggtctttagtctcattagaccaaatgagaatccagcacataagagtcaattagtgtggatggcatgccactctgcagcatttgaggaccttagagtctcaagtttcatcagagggacaagagtggtcccaagaggacagctatccaccagaggggttcaaattgcttcaaatgagaacatggaagcaatggactccaacactcttgaactgagaagtagatattgggctataagaaccagaagcggaggaaacaccaaccagcagagggcatctgcaggacagatcagcgttcagcccactttctcggtacagagaaaccttcccttcgaaagatcgaccattatggcagcatttacaggaaatactgagggcagaacgtctgacatgaggactgaaatcataagaatgatggaaagtgccagaccagaagatgtgtcattccaggggcggggagtcttcgagctctcggacgaaaaggcaacgaacccgatcgtgccttcctttgacatgaataatgaaggatcttatttcttcggagacaatgcagaggagtatgacaattaaagaaaaatacccttgtttctact

SEQ ID NO: 57 - DNA, 1185 bp, Influenza A virus
atgtctcttcaagggattcacctgagtgatttatcatacaagcatgctatattaaaagagtctcagtacacaataaaaagagatgtgggtacaacaactgcagtgacaccctcatcattgcaacaagaaataacactgttgtgtggagaaattctgtatgctaaacatgctgactacaaatatgctgcagaaataggaatacaatatattagcacagctttaggatcagagagagtgcagcagattctgaggaactcaggcagtgaagtccaagtggtcttaaccagaacgtactctctggggaaaattaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgccttaatattcactaaactagcatcaaccatagaagtgggactagagaccacagtcagaagggctaaccgtgtactaagtgatgcactcaagagataccctagaatggacataccaaagattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggggccggtcaaacaatgctaaggtggggggtcattgccaggtcatccaacaatataatgttaggacatgtatccgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgagggagagtaccaaacacagaattattttcagcagctgaaagttatgccaaaagtttgaaagaaagcaataaaataaatttctcttcattaggacttacagatgaagagaaagaggctgcagaacatttcttaaatgtgagtgacgacagtcaaaatgattatgagtaa

SEQ ID NO: 58 - DNA, 1170 bp, Influenza A virus
atggctacagtcaaatgggctgatgcatctgaaccacaacgtggtcgtcagggtagaataccttattctctttatagccctttgcttgttgatagtgaacaaccttggaaggtgatacctcgtaatttggtacccatcaacaagaaagacaaaaataagcttataggctattggaatgttcaaaaacgtttcagaactagaaagggcaaacgggtggatttgtcacccaagttacatttttattatcttggcacaggaccccataaagatgcaaaatttagagagcgtgttgaaggtgttgtctgggttgctgttgatggtgctaaaactgaacctacaggttacggtgttaggcgcaagaattcagaaccagagataccacacttcaatcaaaagctcccaaatggtgttactgttgttgaagaacctgactcccgtgctccttcccgttctcagtcaaggtctcagagtcgcggtcgtggtgaatccaaatctcaatctcggaatccttcaagtgacagaaaccataacagtcaggatgacatcatgaaggcagtcgctgcggctcttaaatctttaggttttgacaagcctcaggaaaaagacaaaaagtcagcgaaaacgggtactcctaagccttctcgtaatcagagtcctgcttcttctcaatctgttgccaagattcttgctcgttctcagagttctgaaacaaaagaacaaaagcatgaaatgcaaaagccacggtggaaaagacagcctaacgatgatgtgacatctaatgtcacacaatgttttggccccagagaccttgaccacaactttggaagtgcaggtgttgtggccaatggcgttaaagctaaaggctatccacaatttgctgagcttgtgccgtctacagctgctatgctttttgatagtcacattgtttccaaagagtcaggcaacactgtggtcttgactttcaccactagagtgactgtgcccaaagaccatccacacttgggtaagtttcttgaggaattaaatgcattcactagagaaatgcaacaacagcctcttcttaaccctagtgcactagaattcaacccatcccaaacttcacctgcaactgttgaaccagtgcgtgatgaagtttctattgaaactgacataattgatgaagtcaactaa

SEQ ID NO: 59 - protein, 1434 aa, Influenza A virus
MGARASVLSGGELDRWEKIRLRPGGKKKYKLKHIVWASRELERFVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLREDLAFLQGKAREFSSEQTRANSPTRRELQVWGRDNNSPSEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 60 - protein, 55 aa, Influenza A virus
MQRGNFRNQRKIVKCFNCGKEGHTARNCRAPRKKGCWKCGKEGHQMKDCTERQAN

SEQ ID NO: 61 - protein, 132 aa, Influenza A virus
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNY

SEQ ID NO: 62 - DNA, 984 bp, Influenza A virus
atgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcactattgccgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaa

SEQ ID NO: 63 - protein, 121 aa, Influenza A virus
MDPNTVSSFQDILLRMSKMQLESSSGDLNGMITQFESLKLYRDSLGEAVMRMGDLHSLQNRNEKWREQLGQKFEEIRWLIEEVRHKLKITENSFEQITFMQALHLLLEVEQEIRTFSFQLI

SEQ ID NO: 64 - protein, 230 aa, Influenza A virus
MDPNTVSSFQVDCFLWHVRKRVADQELGDAPFLDRLRRDQKSLRGRGSTLGLDIETATRAGKQIVERILKEESDEALKMTMASVPASRYLTDMTLEEMSRDWSMLIPKQKVAGPLCIRMDQAIMDKNIILKANFSVIFDRLETLILLRAFTEEGAIVGEISPLPSLPGHTAEDVKNAVGVLIGGLEWNDNTVRVSETLQRFAWRSSNENGRPPLTPKQKREMAGTIRSEV

SEQ ID NO: 65 - protein, 716 aa, Influenza A virus
MEDFVRQCFNPMIVELAEKTMKEYGEDLKIETNKFAAICTHLEVCFMYSDFHFINEQGESIIVELGDPNALLKHRFEIIEGRDRTMAWTVVNSICNTTGAEKPKFLPDLYDYKENRFIEIGVTRREVHIYYLEKANKIKSEKTHIHIFSFTGEEMATKADYTLDEESRARIKTRLFTIRQEMASRGLWDSFRQSERGEETIEERFEITGTMRKLADQSLPPNFSSLENFRAYVDGFEPNGYIEGKLSQMSKEVNARIEPFLKTTPRPLRLPNGPPCSQRSKFLLMDALKLSIEDPSHEGEGIPLYDAIKCMRTFFGWKEPNVVKPHEKGINPNYLLSWKQVLAELQDIENEEKIPKTKNMKKTSQLKWALGENMAPEKVDFDDCKDVGDLKQYDSDEPELRSLASWIQNEFNKACELTDSSWIELDEIGEDVAPIEHIASMRRNYFTSEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLIRSAIGQVSRPMFLYVRTNGTSKIKMKWGMEMRRCLLQSLQQIESMIEAESSVKEKDMTKEFFENKSETWPIGESPKGVEESSIGKVCRTLLAKSVFNSLYASPQLEGFSAESRKLLLIVQALRDNLEPGTFDLGGLYEAIEECLINDPWVLLNASWFNSFLTHALS

SEQ ID NO: 66 - protein, 757 aa, Influenza A virus
MDVNPTLLFLKVPAQNAISTTFPYTGDPPYSHGTGTGYTMDTVNRTHQYSEKGRWTTNTETGAPQLNPIDGPLPEDNEPSGYAQTDCVLEAMAFLEESHPGIFENSCIETMEVVQQTRVDKLTQGRQTYDWTLNRNQPAATALANTIEVFRSNGLTANESGRLIDFLKDVMESMNKEEMGITTHFQRKRRVRDNMTKKMITQRTMGKKKQRLNKRSYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYMTRNQPEWFRNVLSIAPIMFSNKMARLGKGYMFESKSMKLRTQIPAEMLASIDLKYFNDSTRKKIEKIRPLLIEGTASLSPGMMMGMFNMLSTVLGVSILNLGQKRYTKTTYWWDGLQSSDDFALIVNAPNHEGIQAGVDRFYRTCKLLGINMSKKKSYINRTGTFEFTSFFYRYGFVANFSMELPSFGVSGINESADMSIGVTVIKNNMINNDLGPATAQMALQLFIKDYRYTYRCHRGDTQIQTRRSFEIKKLWEQTRSKAGLLVSDGGPNLYNIRNLHIPEVCLKWELMDEDYQGRLCNPLNPFVSHKEIESMNNAVMMPAHGPAKNMEYDAVATTHSWIPKRNRSILNTSQRGVLEDEQMYQRCCNLFEKFFPSSSYRRPVGISSMVEAMVSRARIDARIDFESGRIKKEEFTEIMKICSTIEELRRQK

SEQ ID NO: 67 - protein, 759 aa, Influenza A virus
MERIKELRNLMSQSRTREILTKTTVDHMAIIKKYTSGRQEKNPALRMKWMMAMKYPITADKRITEMIPERNEQGQTLWSKMNDAGSDRVMVSPLAVTWWNRNGPITNTVHYPKIYKTYFERVERLKHGTFGPVHFRNQVKIRRRVDINPGHADLSAKEAQDVIMEVVFPNEVGARILTSESQLTITKEKKEELQDCKISPLMVAYMLERELVRKTRFLPVAGGTSSVYIEVLHLTQGTCWEQMYTPGGEVRNDDVDQSLIIAARNIVRRAAVSADPLASLLEMCHSTQIGGIRMVDILRQNPTEEQAVDICKAAMGLRISSSFSFGGFTFKRTSGSSVKREEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGVEPIDNVMGMIGILPDMTPSIEMSMRGVRISKMGVDEYSSTERVVVSIDRFLRIRDQRGNVLLSPEEVSETQGTEKLTITYSSSMMWEINGPESVLVNTYQWIIRNWETVKIQWSQNPTMLYNKMEFEPFQSLVPKAIRGQYSGFVRTLFQQMRDVLGTFDTAQIIKLLPFAAAPPKQSRMQFSSFTVNVRGSGMRILVRGNSPVFNYNKATKRLTVLGKDAGTLTEDPDEGTAGVESAVLRGFLILGKEDKRYGPALSINELSNLAKGEKANVLIGQGDVVLVMKRKRDSSILTDSQTATKRIRMAIN

SEQ ID NO: 68 - protein, 97 aa, Influenza A virus
MSLLTEVETPIRNEWGCRCNGSSDPLTIAANIIGILHLTLWILDRLFFKCIYRRFKYGLKGGPSTEGVPKSMREEYRKEQQSAVDADDGHFVSIELE